(19) United States
(12) Patent Application Publication
(10) Pub. No.: US 2018/0318503 A1
(43) Pub. Date: Nov. 8, 2018

(54) Implantable Drug Delivery Device with Infusate Measuring Capabilities

(71) Applicant: Flowonix Medical Incorporated, Mt. Olive, NJ (US)

(72) Inventors: Paul BURKE, Bellingham, MA (US); Shannon O'TOOLE, Mt.Olive, NJ (US); Jeff COLELLA, Mt. Olive, NJ (US)

(21) Appl. No.: 16/039,964

(22) Filed: Jul. 19, 2018

Related U.S. Application Data
(63) Continuation-in-part of application No. 15/098,663, filed on Apr. 14, 2016.
(60) Provisional application No. 62/148,457, filed on Apr. 16, 2015.

(51) Int. Cl. A61M 5/168; A61M 5/155

(52) U.S. Cl. CPC A61M 5/1684; A61M 2205/3375; A61M 5/16881; A61M 5/16886; A61M 2205/3337; A61M 2205/7518; A61M 2205/7536; A61M 2205/505; A61M 2205/50; A61M 2205/15; A61M 2005/16863; A61M 2205/3389; A61M 2205/3561; A61M 2205/18; A61M 2205/3331; A61M 2205/3306; A61M 2205/3317; A61M 5/155

(57) ABSTRACT

An implantable drug delivery device and method that includes a bellows sensor for detecting the displacement of a bellows within an infusate reservoir. Sensor data from the bellows sensor may enable indirect measurement of the flow conditions of the implantable drug delivery device or connected catheter. A processor within the implantable drug delivery device may use the sensor data to determine whether delivery of infusate to a patient over time is outside normal or acceptable parameters and take an action in response.

### RELATED APPLICATION

This application is a continuation-in-part of U.S. patent application Ser. No. 15/098,663 titled “Implantable Drug Delivery Device with Flow Measuring Capabilities” filed on Apr. 14, 2016 which claims the benefit of priority to U.S. Provisional Application No. 62/148,457, entitled “Implantable Drug Delivery Device with Flow Measuring Capabilities” filed on Apr. 16, 2015, the entire contents of all of which are incorporated herein by reference.

### FIELD

The present invention relates generally to implantable infusion devices for the delivery of medication or other fluids to a patient.

### BACKGROUND

Various implantable devices exist for delivering infusate, such as medication, to a patient. One such device is an implantable valve accumulator pump system. This system includes an electronically controlled metering assembly located between a drug reservoir and an outlet catheter. The metering assembly may include two normally closed solenoid valves that are positioned on the inlet and outlet sides of a fixed volume accumulator. The inlet valve opens to admit a fixed volume of infusate from the reservoir into the accumulator. Then, the inlet valve is closed and the outlet valve is opened to dispense the fixed volume of infusate from the accumulator to an outlet catheter through which the infusate is delivered to the patient. The valves may be controlled electronically via an electronics module, which can optionally be programmed utilizing an external programmer to provide a programmable drug delivery rate. Because the device is typically implanted in the patient's body and not easily accessed while it is operating, it can be difficult to detect when there is a fault condition or other deviation from normal operating conditions of the device.

### SUMMARY

The systems, methods, and devices of the various embodiments provide an indirect measurement of the flow rate of an implantable drug delivery device by monitoring the volume of a bellows that provides the reservoir for infusate. The various embodiments may enable monitoring of the flow rate condition of the implantable drug delivery device by measuring the change in shape or displacement of the bellows over time. Various embodiments include an implantable drug delivery device having a bellows sensor configured to measure a change in shape or displacement of the bellows as a function of time. The bellows sensor may be an electronically-based sensor, such as strain gauge or capacitive displacement sensor, a light-based sensor, a pressure sensor or a sonically-based sensor.

### BRIEF DESCRIPTION OF THE DRAWINGS

The accompanying drawings, which are incorporated herein and constitute part of this specification, illustrate example embodiments of the invention, and together with the general description given above and the detailed description given below, serve to explain the features of the invention.

FIG. 1 is a schematic diagram of an implantable drug delivery system.

FIGS. 2A-2D schematically illustrate a fixed-volume accumulator of a metering assembly and the sequence of steps performed by the metering assembly of the implantable drug delivery system.

FIG. 3 is a schematic diagram of an embodiment implantable drug delivery device that includes a strain gauge sensing device configured to measure a change in position or deflection of a diaphragm of an accumulator.

FIG. 4 is a schematic diagram of an embodiment implantable drug delivery device that includes a capacitive displacement sensor configured to measure a change in position or deflection of a diaphragm of an accumulator.

FIG. 5 is a schematic diagram of an embodiment implantable drug delivery device that includes a light-based sensor configured to measure a change in position or deflection of a diaphragm of an accumulator.

FIG. 6 is a schematic diagram of an embodiment implantable drug delivery device that includes a pressure sensor configured to measure a change in position or deflection of a diaphragm of an accumulator.

FIG. 7 a schematic diagram of an embodiment implantable drug delivery device that includes a sonic-based sensor configured to measure a change in position or deflection of a diaphragm of an accumulator.

FIG. 8 is a process flow diagram illustrating a method of operating an implantable drug delivery device according to an embodiment.

FIGS. 9-15 are schematic diagrams of various embodiments of an implantable drug delivery device that include different bellows sensors and/or detectors configured to measure one or more parameters associated with the volume of infusate and/or the displacement of the bellows.

FIG. 16 is a process flow diagram illustrating an embodiment method for determining whether a change in infusate volume within a bellows of an implantable drug delivery device according to some embodiments.

FIG. 17 is a process flow diagram illustrating an embodiment method for an example abnormal infusate volume rate procedure.

FIG. 18 is a process flow diagram illustrating an embodiment method for determining initial parameters associated with a bellows of an implantable drug delivery device.

FIG. 19 is a process flow diagram illustrating an embodiment method for taking actions based on changes in flow-related parameters associated with infusate within a bellows of an implantable drug delivery device.

FIG. 20 is schematic diagram of another implantable drug delivery system according to an embodiment.

### DETAILED DESCRIPTION

The various embodiments will be described in detail with reference to the accompanying drawings. Wherever possible, the same reference numbers will be used throughout the drawings to refer to the same or like parts. References made to particular examples and implementations are for illustrative purposes, and are not intended to limit the scope of the invention or the claims.

The words “exemplary” or “for example” are used herein to mean “serving as an example, instance, or illustration.” Any implementation described herein as “exemplary” or “for example” is not necessarily to be construed as preferred or advantageous over other implementations.

The systems, methods, and devices of the various embodiments enable monitoring doses of an infusate to a patient by monitoring displacement of the bellows that provides the reservoir for the infusate. An embodiment drug delivery system may include a bellows sensor configured to measure a change in displacement or shape of the bellows. The bellows sensor may be, for example, an electronically-based sensor, such as a strain gauge or capacitive displacement sensor, a light-based sensor, a pressure sensor, or a sonically-based sensor. The bellows sensor may be used to provide an indirect measurement of the flow rate of an implantable drug delivery device by monitoring the change in volume of the bellows over time. The various embodiments may enable a determination of whether or not the flow rate of the implantable drug delivery device is within normal operating conditions by measuring the change in shape or displacement of the bellows as a function of time.

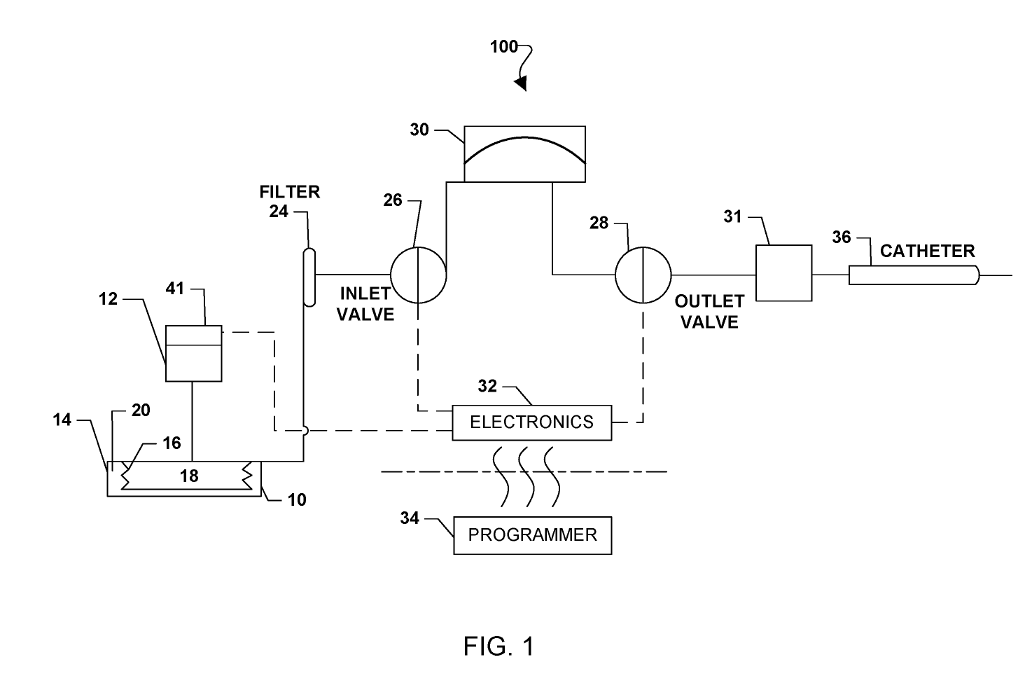

FIG. 1 illustrates an embodiment of an implantable valve accumulator pump system 100 for the delivery of infusate, such as medication. The system 100 may generally include four assemblies. The first major assembly is a rechargeable, constant pressure drug reservoir 10 in series with a bacteria/air filter 24. In one embodiment, the reservoir 10 includes a sealed housing 14 containing a bellows 16. The bellows 16 separates the housing 14 into two parts, a chamber 18 and a second zone 20. The chamber 18 is used to hold the drug or other medicinal fluid. The second zone 20 is normally filled with a two-phase fluid, such as Freon®, that has a significant vapor pressure at body temperature. Thus, as the fluid within the second zone 20 vaporizes, the vapor compresses the bellows 16, thereby pressurizing the drug in the chamber 18. The chamber 18 can be refilled with an infusate via a refill septum 12.

The two-phase fluid helps maintain the chamber 18 under a constant pressure. When the chamber 18 is refilled, the two-phase fluid is pressurized thereby condensing a portion of the vapor within the second zone 20 to the liquid phase. As the chamber 18 is emptied, this liquid vaporizes, thus maintaining the pressure on the bellows 16. Since the infusate in the chamber 18 is under positive pressure, the infusate is urged out of the chamber through a bacterial filter 24 and toward the metering assembly.

The second major assembly is an electronically controlled metering assembly that may include two normally closed solenoid valves 26, 28 that are positioned on the inlet and outlet sides of a fixed volume accumulator 30. The valves are controlled electronically via an electronics module 32, which may be programmed utilizing the external programmer 34. The metering assembly may be designed such that the inlet valve 26 and the outlet valve 28 are never simultaneously open.

The third major assembly is an outlet catheter 36 for medication infusion in a localized area. The delivery of fluid occurs at an infusion site that has a pressure less than the accumulator pressure. This pressure difference forces discharge of the infusate through the catheter 36.

The drug reservoir 10 and electronically controlled metering assembly may be contained within a biocompatible housing, also containing a power source (e.g., battery) that may be implanted within the body of a human or animal patient. The outlet catheter 36 may be integral with the housing, or may be a separate component that is attached to the housing. An access port 31, in communication with the catheter 36, may be provided downstream of the metering assembly. The access port 31 may be used, for example, to manually provide a bolus dose of medication to the patient.

The fourth assembly of the system illustrated in FIG. 1 is an external programmer 34 used to communicate and program the desired medication regimen. In an embodiment, the external programmer 34 may be a handheld unit with a touch screen. The external programmer 34 may provide a wireless data transfer link to a wireless communication transceiver within the implanted electronics module 32 and may be enabled to exchange information with the electronic module 32, including but not limited to battery status, diagnostic information, calibration information, etc. In various embodiments described in further detail below, the electronic module 32 may communicate information regarding the flow rate of infusate from the implantable system 100 to the external programmer 34. In an embodiment, the external programmer 34 may send an instruction to the electronics module 32 to detect the flow rate of infusate from the implantable system according to the embodiments described below. In an embodiment, the electronics module 32 may include a coil configured to send and receive electromagnetic signals to/from the external programmer 34.

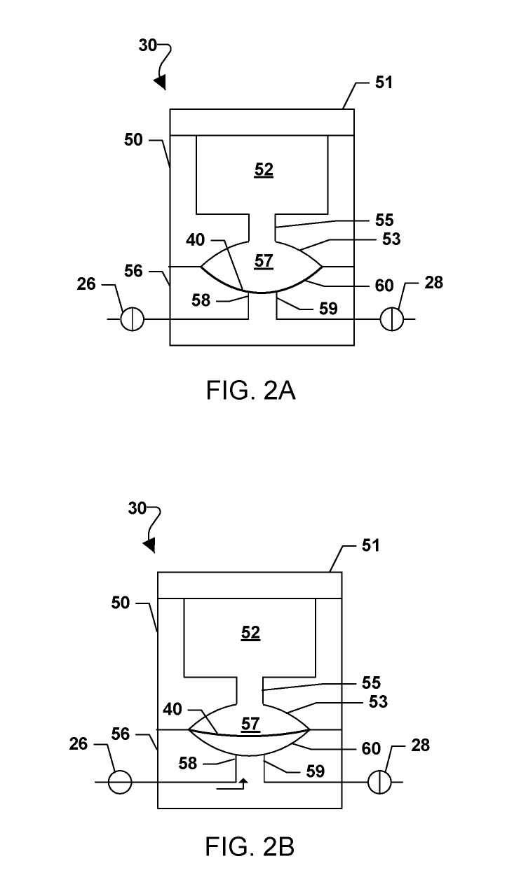

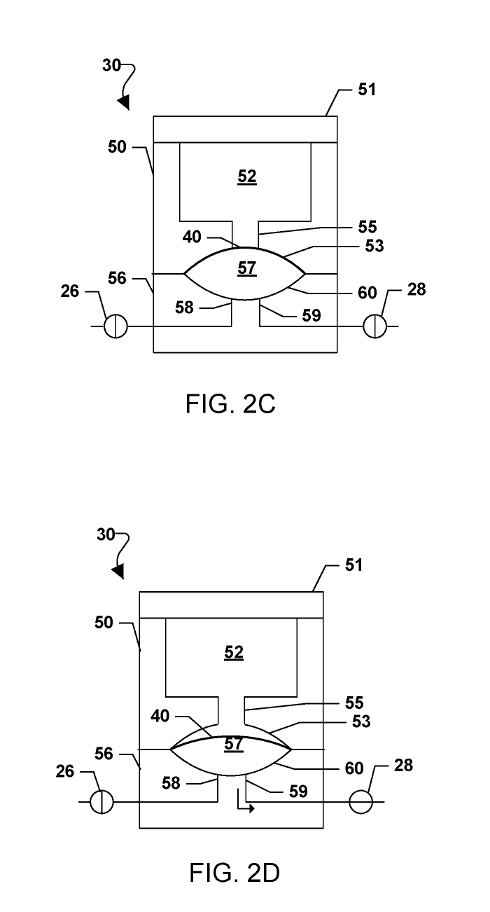

FIGS. 2A-2D schematically illustrate the structure and operation of a fixed volume accumulator 30 of an electronically-controlled metering assembly according to one embodiment. The accumulator 30 may include a housing 50 that together with a cap 51 defines a sealed gas chamber 52. The cap 51 may be secured to the housing 50 using any suitable means, such as laser welding. A suitable gas may be sealed, under positive pressure, within the gas chamber 52. The sealed gas chamber 52 may contain an inert gas such as argon, helium or nitrogen, air, or mixtures of different gases. Alternately, the sealed gas chamber 52 may contain a two-phase fluid. A bottom surface of the housing 50 may define a first (e.g., upper) surface 53 of a diaphragm chamber 57. One or more fluid passages 55 within the housing 50 may connect the gas chamber 52 with the diaphragm chamber 57.

A face plate 56 (which may also be referred to as a spacer plate) may be secured to the bottom surface of the housing 50. An upper surface of the face plate 56 may define a second (e.g., lower) surface 60 of the diaphragm chamber 57. A diaphragm 40 may be located between the housing 50 and the face plate 56 and within the diaphragm chamber 57 defined therebetween. In embodiments, the edges of the diaphragm 40 may be sandwiched between the housing 50 and the face plate 56, and the assembly may be sealed, such as via laser welding. The diaphragm 40 may provide a barrier separating a gas side (e.g., above the diaphragm 40) from a fluid side (e.g., below the diaphragm 40) in the accumulator 30. The face plate 56 may include a fluid inlet port 58 that provides fluid communication between the inlet valve 26 and the diaphragm chamber 57 and a fluid outlet port 59 that provides fluid communication between the outlet valve 28 and the diaphragm chamber 28.

In embodiments, the diaphragm 40 may include a thin, disk-shaped sheet. The diaphragm 40 may include a metal, such as titanium. The diameter and thickness of the diaphragm 40 may be selected to provide a low spring rate over a desired range of deflection. The diaphragm 40 may function as a compliant, flexible wall that separates a fluid (e.g., liquid infusate) from the environment behind it. In the embodiment illustrated in FIGS. 2A-2B, the deflections of the diaphragm 40, illustrated as upward and downward motions, are limited by the first and second surfaces 53, 60 of the diaphragm chamber 57 that act as mechanical stops for the diaphragm 40. In the embodiment illustrated in FIGS. 2A-2B, each of these surfaces 53, 60 are formed having a shallow concave profile that acts as a contour stop for the diaphragm 40. The dimensions of the contour may be chosen to match the general profile of the diaphragm 40 when it is deflected or biased by a predetermined fixed volume. This predetermined fixed volume corresponds to the volume that is metered by the accumulator 30. In other embodiments, one of the surfaces 53, 60 may have a generally flat profile that corresponds to the profile of the diaphragm in a flat, undeflected state, while the other surface may correspond to the profile of the diaphragm in a deflected state.

In some embodiments, the second (e.g., lower) surface 60 of the diaphragm chamber 57 may include one or more channels formed in the surface 60 to maximize wash out of fluid and minimize dead volume within the chamber 57. For example, the surface 60 may be formed with an annular groove intersected by a trough connecting the inlet and outlet ports 58, 59, such as described in U.S. Pat. No. 8,273,058 to Burke et al., which is incorporated herein by reference for details of the diaphragm chamber.

FIG. 2A illustrates the accumulator 30 in a state in which both the inlet valve 26 and the outlet valve 28 are closed, and the diaphragm 40 deflects downward (in the orientation presented in FIG. 2A) as a result of the bias from the gas pressure in the gas chamber 52 and in the gas side of the diaphragm chamber 57. In this portion of the pumping cycle, there is no liquid infusate in the diaphragm chamber 57.

FIG. 2B shows the accumulator 30 after the inlet valve 26 is opened, while the outlet valve 28 remains closed. The pressure of the liquid infusate from reservoir 10 (see FIG. 1) is sufficient to overcome the bias of the pressurized gas against the back side of the diaphragm 40, causing the diaphragm 40 to separate from the second (lower) surface 60 of the diaphragm chamber 57. The infusate begins to flow into the diaphragm chamber 57 through the inlet port 58, as indicated by the arrow in FIG. 2B. As the infusate fills the diaphragm chamber 57, the bias from the fluid pressure in the chamber 57 causes the diaphragm 40 to deflect upwards (in the orientation presented in FIG. 2B) towards the first (upper) surface 53 of the diaphragm chamber 57.

FIG. 2C shows the accumulator 30 filled with infusate to its fixed or desired volume. The diaphragm 40 is biased against the first (upper) surface 53 of the diaphragm chamber 57, which acts as a mechanical stop for the diaphragm 40. When the accumulator 30 is filled with infusate, the inlet valve 26 is closed, as shown in FIG. 2C.

FIG. 2D shows the accumulator 30 after the outlet valve 28 is opened while the inlet valve 26 remains closed. The infusate begins to flow out of the diaphragm chamber 57 through the outlet port 59 and the catheter 30 (see FIG. 1), as indicated by the arrow in FIG. 2D. As the infusate empties the accumulator, the diaphragm 40 separates from the first (upper) surface 53 of the diaphragm chamber 57. The bias from the gas pressure in the gas chamber 52 and in the gas side of the diaphragm chamber 57 causes the diaphragm 40 to deflect downwards (in the orientation presented in FIG. 2D) towards the second (lower) surface 60 of the diaphragm chamber 57. When the chamber 57 is completely emptied of infusate, the diaphragm 40 is biased against the second (lower) surface 60 of the diaphragm chamber 57, which acts as a mechanical stop for the diaphragm 40. The outlet valve 28 is then closed and the accumulator 30 is again in the state shown in FIG. 2A. The pumping cycle illustrated in FIGS. 2A-2D may then be repeated. The accumulator 30 thus stores and discharges predetermined volume spikes of infusate at a frequency defined by the cycling rate of the inlet and outlet valves 26, 28 of the accumulator 30. The nominal flow rate of infusate from the system 100 may be controlled by controlling the cycling rate of the inlet and outlet valves 26, 28 of the accumulator 30.

In operation, the programmed flow rate of infusate from the system may not represent the actual rate of infusate being delivered to the patient for a variety of reasons. For example, there may be a blockage or occlusion of the infusate flow in the catheter or elsewhere in the device, a malfunctioning valve, a leak in the device, or another fault condition. Any one or combination of these conditions may result in a situation in which more or less than the desired amount of the infusate is being delivered to the patient in a given time period. This can result in reduced efficacy of the treatment regimen and can potentially be dangerous to the patient. Further, it has generally not been possible to directly measure the amount of infusate being delivered to the patient from the catheter (e.g., using a conventional fluid flow meter) since the infusate is typically delivered to a confined and sensitive area inside the patient's body where the use of conventional flow meters is impractical.

The various embodiments include methods and systems for indirectly measuring the flow rate of an implantable drug delivery device by measuring the movement of a diaphragm in a fixed-volume accumulator. Embodiments include various systems and methods for measuring a change in position or deflection of the diaphragm over time to determine the rate of flow of infusate from the accumulator. For example, referring to the fixed volume accumulator 30 illustrated in FIGS. 2A-2D, the amount of time it takes for the diaphragm 40 to move from the position shown in FIG. 2C (i.e., with the diaphragm biased against the first (upper) surface 53 of the diaphragm chamber 57) to the position shown in FIG. 2A (e.g., with the diaphragm biased against the second (lower) surface 60 of the diaphragm chamber 57) is directly related to the flow rate of the known volume of infusate that is dispensed from the accumulator during a pumping cycle. This time may vary based on the amount of flow restriction in the catheter or elsewhere in the system. In some cases, such as when there is a blockage or leak in the flow path of the device, the diaphragm chamber 57 may not completely fill or discharge during each pumping cycle (e.g., such that the diaphragm does not fully deflect to the positions illustrated in FIGS. 2A and/or 2C during the pumping cycle). This may be detected by measuring the change in position or deflection of the diaphragm as a function of time.

Various embodiments may include an implantable drug delivery device that includes a diaphragm sensor for detecting a change in position or deflection of a diaphragm of a fixed volume accumulator. An electronics module connected to the diaphragm sensor may monitor the detected change in position or deflection of the diaphragm as a function of time to determine whether the flow rate of the device satisfies at least one pre-determined criteria. The electronics module may be configured such that in response to determining that the flow rate does not satisfy the pre-determined criteria, the electronics module may take an appropriate action, such as sending a wireless signal providing a notification to a user of the device and/or medical personnel, adjusting the cycling rate of the fixed-volume accumulator to bring the flow rate within the pre-determined criteria, and/or shutting down the device to prevent further infusion of the medication.

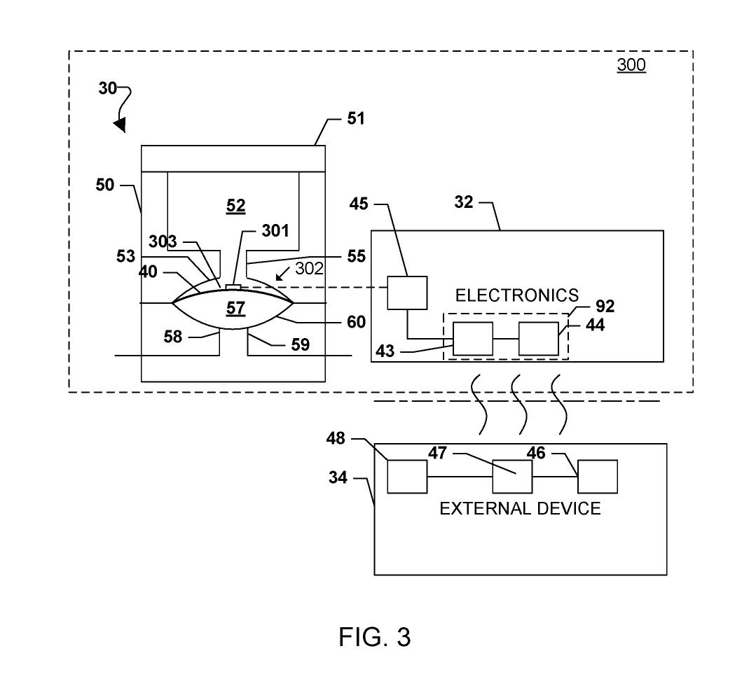

The diaphragm sensor may be any suitable diaphragm sensor that is configured to detect a change in position or deflection of the diaphragm 40. FIG. 3 illustrates a first embodiment of an implantable drug delivery device 300 that includes an electronically-based diaphragm sensor 302 configured to measure a change in position or deflection of a diaphragm 40 of an accumulator 30 as a function of time. In this embodiment, the electronically-based diaphragm sensor 302 may include at least one strain gauge 301. The at least one strain gauge 301 may be located on a surface 303 of the diaphragm 40 that is exposed to the gas from the sealed gas chamber 52 and opposite the surface of the diaphragm 40 that is exposed to the infusate (the surface 303 may alternately be referred to as the “back side” of the diaphragm 40). Alternatively or in addition, one or more strain gauges may be located on the “front side” of the diaphragm (i.e., the surface that is exposed to the infusate in the diaphragm chamber 57).

The at least one strain gauge 301 may include any suitable type of diaphragm sensor device for converting mechanical strain to a proportional electrical signal. For example, the at least one strain gauge 301 may include a bonded foil strain gauge, a bonded semiconductor strain gauge (e.g., a piezoresistor), a thin film strain gauge (e.g., a strain gauge formed by vapor deposition or sputtering of an insulator and gauge material onto the surface of the diaphragm), and/or a diffused or implanted semiconductor strain gauge. The at least one strain gauge may be calibrated to measure the strain corresponding to the displacement (i.e. deflection) of the diaphragm 40 between a flat, resting-state position to the maximum upward and/or downward deflection positions of the diaphragm 40 within the accumulator 30 (i.e., the positions of the diaphragm shown in FIGS. 2A and 2C).

In the device 300 illustrated in FIG. 3, the electronics module 32 may include a controller 92. In an embodiment, the controller 92 may include a processor 43 coupled to a memory 44. The processor 43 may be any type of programmable processor, such as a microprocessor or microcontroller, which may be configured with processor-executable instructions to perform the operations of the embodiments described herein. Processor-executable software instructions may be stored in the memory 44 from which they may be accessed and loaded into the processor 43. The processor 43 may include internal memory sufficient to store the application software. The memory 44 may be volatile, nonvolatile such as flash memory, or a mixture of both.

In an embodiment, the controller 92 may be coupled to a strain gauge monitoring circuit 45 of the diaphragm sensor 302. The strain gauge monitoring circuit 45 may measure a change in an electrical characteristic (e.g., resistance) of the at least one strain gauge 301 corresponding to the strain experienced by the strain gauge 301. The strain gauge monitoring circuit 45 may include a four-gauge Wheatstone bridge circuit, for example. The electronics module 32 may also include a clock generator that generates timing signals so that each of the measured strain values may be associated with a particular measurement time. The controller 92 may compare the measured strain from the monitoring circuit 45 to pre-determined strain values corresponding to different deflection positions of the diaphragm 40 within the accumulator 30. The pre-determined strain values may be stored in the memory 44, such as in the form of a look-up table, for example. The controller 92 may use the measured strain values from the monitoring circuit 45 and the known pre-determined values corresponding to different deflection positions of the diaphragm 40 to determine the change in position or deflection of the diaphragm 40 (i.e., the amount of upward and/or downward deflection of the diaphragm 40 as oriented in the figures) as a function of time. As discussed above, the change in position or deflection of the diaphragm as a function of time may be directly related to the rate at which the infusate is pumped from the accumulator. The controller 92 may be configured to determine whether the detected change in position or deflection of the diaphragm as a function of time is within normal operating parameters (i.e., the detected change of position or deflection of the diaphragm as a function of time corresponds to a clinically acceptable flow rate of the infusate). In some embodiments, the controller 92 may not translate the measured strain values into deflection values, and instead may be configured to determine whether the detected change in measured strain values over a period of time is within normal operating parameters (i.e., the detected change in measured strain values over time corresponds to a clinically acceptable flow rate of the infusate).

The controller 92 may be configured to provide a notification to the user, such as by sending a message to an external device 34, when the detected motion of the diaphragm is determined to be outside normal operating parameters (i.e., not within such parameters). The external device 34 may be a programmer as described above, or alternately another external device may be configured to communicate with the implantable device 300 via a wireless data transfer link.

In various embodiments, the external device 34 may include a processor 47 coupled to a memory 46 and to an indicator 48. Software instructions may be stored in the memory 46 before they are accessed and loaded into the processor 47. The processor 47 may be configured to activate the indicator 48 to provide a notification (e.g., an alarm) to the user when the external device 34 receives a message from the controller 92 of the implantable device 300 indicating that the detected motion of the diaphragm and/or the flow rate of infusate is not within pre-determined parameters. The indicator 48 may be a display, a speaker for an audio or sound message, and/or a vibrator to generate haptic feedback, for example. The processor 47 of the external device 34 may also be configured to notify medical personnel who may be located remotely, such as via a wireless communication network, in response to receiving messages from the controller 92 of the implantable device 300.

In some embodiments, the controller 92 of the implantable device 300 may be configured to detect the motion of the diaphragm on a pre-determined and/or periodic basis (e.g., every hour, every 12 hours, etc.). The scheduled times and/or frequency in which the controller 92 detects the motion of the diaphragm may be varied based on instructions received from the external device 34. Alternatively or in addition, the controller 92 of the implantable device 300 may detect the motion of the diaphragm “on demand” in response to a request or command from the external device 34. In some embodiments, the controller 92 of the implantable device 300 may be configured to detect the motion of the diaphragm 40 continuously or frequently over the duration of a treatment regimen.

In some embodiments, the controller 92 of the implantable device 300 may forward a plurality of raw measurements from the strain gauge monitoring circuit 45 to the external device 34. The processor 47 of the external device 34 may use the raw measurement values to determine the change in diaphragm position or deflection over time and/or the flow rate of infusate from the device 300. The processor 47 of the external device 34 may compare the calculated value(s) to one or more stored threshold values to determine whether the flow rate is within clinically acceptable parameters. In other embodiments, the controller 92 of the implantable device 300 may determine an infusate flow rate value based on the detected change in diaphragm position or deflection over time, and may forward the determined infusate flow rate to the external device 34. The external device 34 may display the flow rate value on the indicator 48.

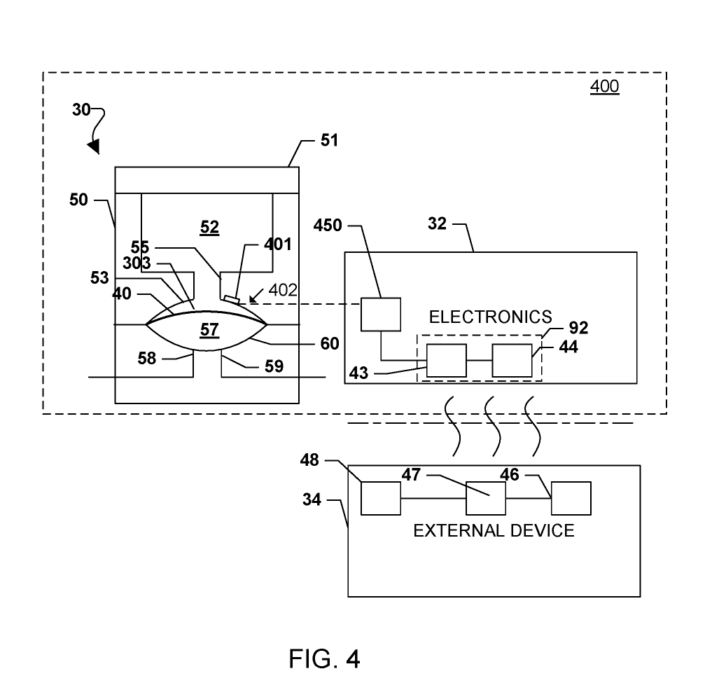

FIG. 4 illustrates a second embodiment of an implantable drug delivery device 400 that includes an electronically-based diaphragm sensor 402 configured to measure a change in position or deflection of a diaphragm 40 of an accumulator 30 as a function of time. In this embodiment, the electronically-based diaphragm sensor 402 may include at least one capacitive displacement sensor 401. Capacitive displacement sensors are noncontact devices that are configured to measure the capacitance between a probe 401 (e.g., an electrode surface) and a target conductive surface (e.g., the surface 303 of the diaphragm 40). The areas of the probe 401 and target surface 303 and the dielectric constant of the material (e.g., gas) between the probe 401 and target surface 303 may be considered constant, in which case the capacitance between the probe 401 and the target surface 303 is proportionally related to the distance between the probe 401 and the target surface 303. Due to this proportional relationship, the diaphragm sensor 402 may measure changes in capacitance as the target surface 303 moves with respect to the probe 402, and a processor may use the measured changes to calculate distance measurements, such as a relative change in the separation distance.

In the embodiment illustrated in FIG. 4, the probe 401 is located proximate to the first (upper) surface 53 of the diaphragm chamber 57, and is configured to measure the displacement of the diaphragm 40 from the first (upper) surface 53 of the chamber 57. Alternatively or in addition, at least one probe 401 may be located proximate to the second (lower) surface 60 of the diaphragm chamber 57 and may be configured to measure the displacement of the diaphragm 40 from the second (lower) surface 60. In other embodiments, a probe 401 may be located on the diaphragm 40 configured to measure the distance between the diaphragm 40 and at least one surface 53, 60 of the diaphragm chamber 57 as the diaphragm moves (i.e., deflects).

The implantable drug delivery device 400 of the embodiment illustrated in FIG. 4 may be similar to the device 300 described above with reference to FIG. 3, and may include an electronics module 32 having a controller 92 comprising a processor 43 and memory 44 as described above. The controller 92 may be coupled to a capacitance monitoring circuit 450 connected to the probe 401 and configured to measure the capacitance between the probe 401 and the surface 303 of the diaphragm 40 as the diaphragm 40 moves within the chamber 57. The controller 92 may be configured to determine changes in the position or deflection of the diaphragm 40 over time based on changes in the measured capacitance. As discussed above, the change in position or deflection of the diaphragm as a function of time may be directly related to the rate at which the infusate is pumped from the accumulator. The controller 92 may be configured to determine whether the detected change in position or deflection of the diaphragm over a period of time is within normal operating parameters (i.e., the detected change of position or deflection of the diaphragm as a function of time corresponds to a clinically acceptable flow rate of the infusate). In some embodiments, the controller 92 may not translate capacitance measurements into distance values, and instead may be configured to determine whether the detected change in capacitance over a period of time is within normal operating parameters (i.e., the detected change in capacitance over time corresponds to a clinically acceptable flow rate of the infusate).

When the detected motion of the diaphragm (or changes in capacitance) is determined to be not within normal operating parameters, the controller 92 may be configured to provide a notification to the user, such as by sending a message to an external device 34. The operation of the device 400 of the embodiment illustrated in FIG. 4 may be substantially similar to the device 300 as described above.

In addition to a mechanical strain gauge and/or capacitive displacement diaphragm sensor as described above, other electronically-based diaphragm sensors may be used to detect the change in position or deflection of the diaphragm 40 as a function of time. For example, the electronically-based diaphragm sensor according to various embodiments may include an eddy current diaphragm sensor and/or an inductive displacement diaphragm sensor.

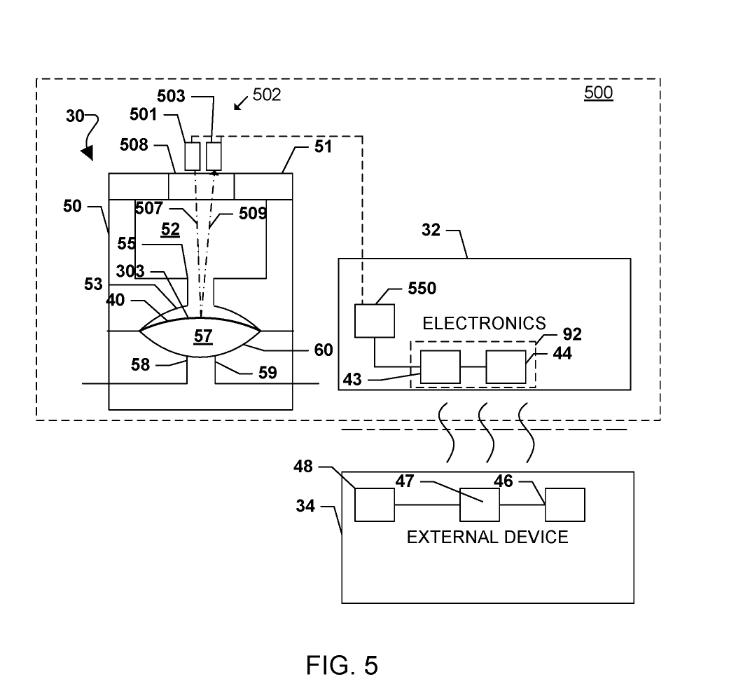

FIG. 5 illustrates a third embodiment of an implantable drug delivery device 500 that includes a light-based diaphragm sensor 502 configured to measure a change in position or deflection of a diaphragm 40 of an accumulator 30 as a function of time. Various devices are known for measuring distance using light signals. A light-based distance measuring device may include a light source 501 (e.g., a laser, LED, etc.) that transmits a beam 507 of radiation (e.g., visible light, UV and/or IR radiation) that is reflected off of a target. The reflected beam 509 is received by a light diaphragm sensor 503 (e.g., a photodiode sensor, a charged coupled device (CCD) sensor, a CMOS-based light sensor, etc.). The distance to the reflective target may be determined using one or more known techniques, such as triangulation, time-of-flight, phase shift, interferometry, chromatic confocal methods, etc. In the embodiment illustrated in FIG. 5, the light beam is reflected off a surface 303 of the diaphragm 40 as the diaphragm 40 deflects within the accumulator 30, and the light-based diaphragm sensor 502 detects the change in position or deflection of the diaphragm 40 over time.

In the embodiment illustrated in FIG. 5, the light source 501 may be located outside of the housing 50 of the accumulator 30 and direct the beam 507 through a transparent window 508 provided in the cap 51 of the housing 50. The beam 507 may be directed through the sealed gas chamber 52 and passage 55 into the diaphragm chamber 57, where the beam 507 is reflected off of the surface 303 of the diaphragm 40. The diaphragm 40 may have a mirror surface 303 to enhance the reflection of the beam. The reflected beam 509 may travel through the passage 55, gas chamber 52 and window 508 and be detected by a light sensor 503 that is located outside of the housing 50 of the accumulator 30. Various other configurations for a light-based diaphragm sensor for measuring displacement of a diaphragm in a fixed-volume accumulator may be used. For example, the light source 501 and/or light sensor 503 may be located within the housing 50, such as within the sealed gas chamber 52, or may be located within the diaphragm chamber 57 (e.g., within surfaces 53 or 60).

The embodiment implantable drug delivery device 500 shown in FIG. 5 may be similar to the devices 300 and 400 described above, and may include an electronics module 32 having a controller 92 comprising a processor 43 and memory 44, as described above. The electronics module 32 may also include a light sensor control circuit 550 coupled to the light source 501 and the light sensor 503 for controlling the operation of the source 501 and light sensor 503 and for generating an electronic signal representation of the reflected light radiation received at the light sensor 503. The controller 92 may be coupled to the light sensor control circuit 550 and may determine changes in the position or deflection of the diaphragm 40 over time based on the electronic signal representation of the reflected light radiation received at the light sensor 503. The controller 92 may use any of the methods described above, including without limitation triangulation, time-of-flight, phase shift, interferometry, and chromatic confocal techniques, to determine the change in position or deflection of the diaphragm 40 over time. As discussed above, the change in position or deflection of the diaphragm as a function of time may be directly related to the rate at which the infusate is pumped from the accumulator. The controller 92 may be configured to determine whether the detected change in position or deflection of the diaphragm as a function of time is within normal operating parameters (i.e., the detected change of position or deflection of the diaphragm as a function of time corresponds to a clinically acceptable flow rate of the infusate). In some embodiments, the controller 92 may not translate measurements from the light sensor into distance values, and instead may be configured to determine whether the detected changes in measured light characteristics (e.g., time of flight, phase shift, interference, etc.) over a period of time are within normal operating parameters (i.e., the detected changes in measured light characteristics over time correspond to a clinically acceptable flow rate of the infusate).

When the detected motion of the diaphragm is determined to be not within normal operating parameters, the controller 92 may be configured to provide a notification to the user, such as by sending a message to an external device 34. The operation of the device 500 may be substantially similar to the operation of the devices 300 and 400 as described above.

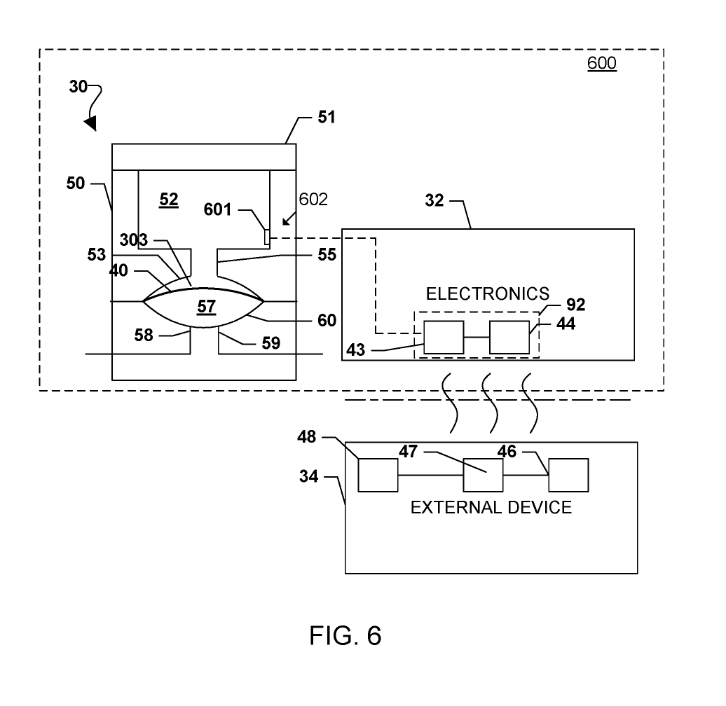

FIG. 6 illustrates a fourth embodiment of an implantable drug delivery device 600 that includes a pressure sensor 602 configured to measure a change in pressure that is related to a change in position or deflection of a diaphragm 40 of an accumulator 30 as a function of time. The pressure sensor 602 may include a pressure transducer 601 that may be located within or in fluid communication with the sealed gas chamber 52 of the accumulator 30. The pressure transducer 602 may be calibrated to detect small changes in the fluid pressure within the chamber 52 as the diaphragm 40 deflects within the diaphragm chamber 57 and may output an electronic signal representing the detected pressure.

The embodiment implantable drug delivery device 600 shown in FIG. 6 may be similar to the devices 300, 400 and 500 described above, and may include an electronics module 32 having a controller 92 comprising a processor 43 and memory 44, as described above. The controller 92 may be coupled to the pressure sensor 602, and may be configured to compare the pressures measured by the pressure sensor 602 to pre-determined pressure values corresponding to different deflection positions of the diaphragm 40 within the accumulator 30. The pre-determined pressure values may be stored in the memory 44 in the form of a look-up table, for example. The controller 92 may use the measured pressure values and the known pre-determined pressure values corresponding to different deflection positions of the diaphragm 40 to determine the change in position or deflection of the diaphragm 40 (i.e., the amount of upward and/or downward deflection of the diaphragm 40) as a function of time. As discussed above, the change in position or deflection of the diaphragm as a function of time may be directly related to the rate at which the infusate is pumped from the accumulator. The controller 92 may be configured to determine whether the detected change in position or deflection of the diaphragm as a function of time is within normal operating parameters (i.e., the detected change of position or deflection of the diaphragm as a function of time corresponds to a clinically acceptable flow rate of the infusate). In some embodiments, the controller 92 may not translate pressure measurements into distance or deflection values, and instead may be configured to determine whether the detected change in pressure over a period of time is within normal operating parameters (i.e., the detected change in pressure over time corresponds to a clinically acceptable flow rate of the infusate).

When the detected motion of the diaphragm is determined to be not within normal operating parameters, the controller 92 may be configured to provide a notification to the user, such as by sending a message to an external device 34. The operation of the device 600 may be substantially similar to the operation of the devices 300, 400 and 500 as described above.

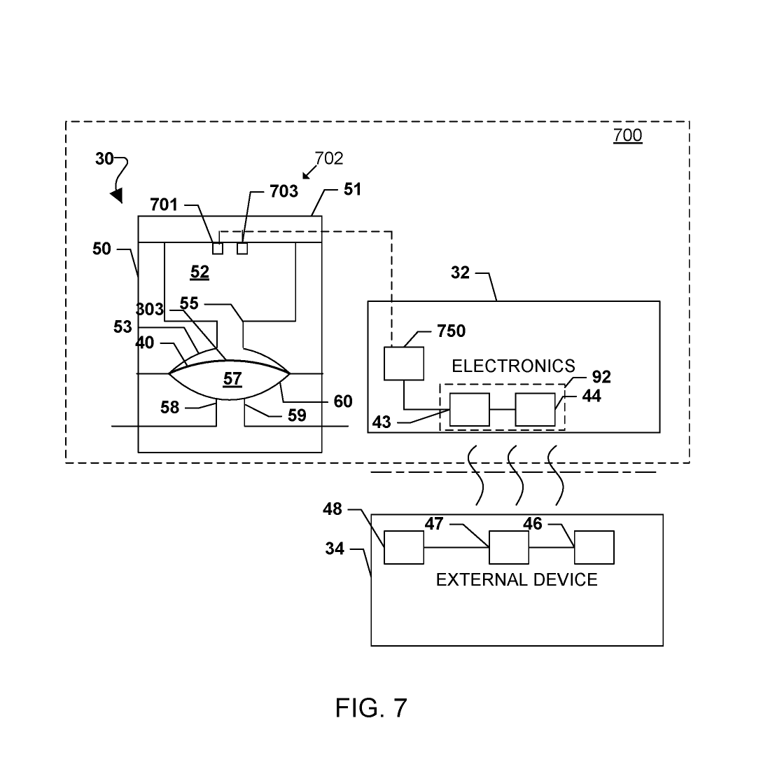

FIG. 7 illustrates a fifth embodiment of an implantable drug delivery device 700 that includes a sonically-based diaphragm sensor 702 configured to measure a change in position or deflection of a diaphragm 40 of an accumulator 30 as a function of time. Various techniques may be used for measuring the displacement of the diaphragm 40 using sonic signals. For example, a source 701 of sonic energy (e.g., a sonic transducer) may generate an acoustic signal (e.g., within an audible, ultrasonic or infrasonic range) within the sealed gas chamber 52 as shown in FIG. 7, or alternatively within the diaphragm chamber 57 (either above or below the diaphragm 40). As the diaphragm deflects within the diaphragm chamber 57, the fluid volume both above and below the diaphragm varies. This variation in volume may change one or more characteristics of the acoustic signal, such a harmonic frequency of the signal, in a manner that may be detected by a sonic sensing device 703. The source 701 of sonic energy and the sonic sensing device 703 are shown as separate devices in FIG. 7, although it will be understood that a single component (e.g., a transducer) may be used to both transmit a sonic energy pulse and receive a reflected pulse (e.g., echo).

The embodiment implantable drug delivery device 700 shown in FIG. 7 may be similar to the devices 300, 400, 500 and 600 described above, and may include an electronics module 32 having a controller 92 including a processor 43 and memory 44, as described above. The electronics module 32 may also include a sonic sensor control circuit 750 coupled to the sonic source 701 and sensing device 703 for controlling the operation of the source 701 and the sensing device 703 and for generating an electronic signal representation of the sonic signal received at the sensing device 703. The controller 92 may be coupled to the sonic sensor control circuit 750 and may determine changes in the position or deflection of the diaphragm 40 over time based on the electronic signal representation of the sonic signal received at the sensing device 703. As discussed above, the change in position or deflection of the diaphragm as a function of time may be directly related to the rate at which the infusate is pumped from the accumulator. The controller 92 may be configured to determine whether the detected change in position or deflection of the diaphragm as a function of time is within normal operating parameters (i.e., the detected change of position or deflection of the diaphragm as a function of time corresponds to a clinically acceptable flow rate of the infusate). In some embodiments, the controller 92 may not translate changes in the received sonic signal into distance values, and instead may be configured to determine whether the detected changes in received sonic signals over a period of time is within normal operating parameters (i.e., the detected changes in sonic signals over time correspond to a clinically acceptable flow rate of the infusate).

When the detected motion of the diaphragm is determined to be not within normal operating parameters, the controller 92 may be configured to provide a notification to the user, such as by sending a message to an external device 34. The operation of the device 700 may be substantially similar to the operation of the devices 300, 400, 500 and 600 as described above.

Various sonically-based diaphragm sensors may be used to detect the change in position or deflection of the diaphragm 40 as a function of time. For example, a sonically-based diaphragm sensor according to various embodiments may use a Doppler, pulse echo and/or sonar technique to measure the displacement of the diaphragm 40 over time.

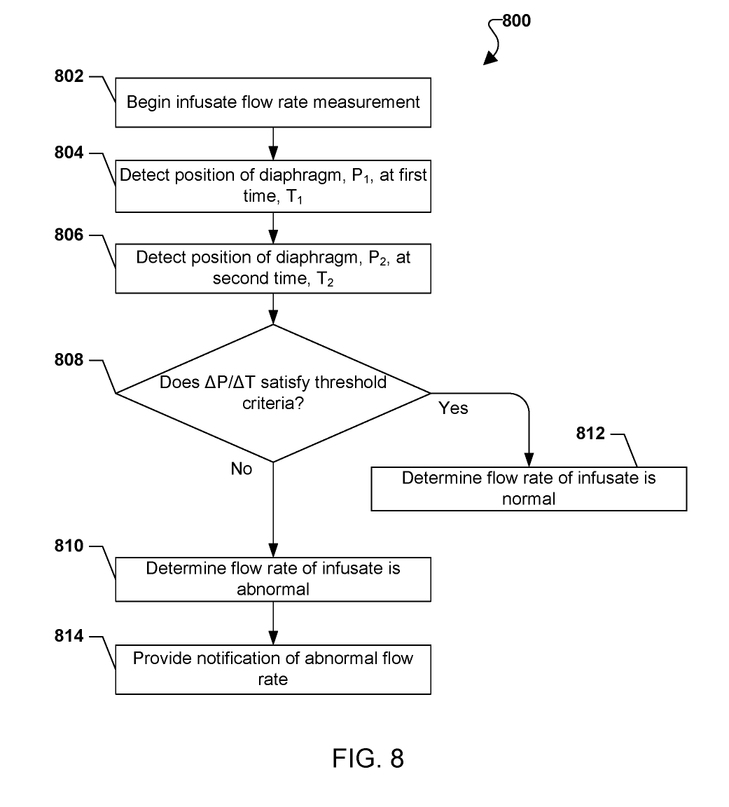

FIG. 8 illustrates an embodiment method 800 for monitoring the flow rate of infusate from an implantable drug delivery device by measuring the movement of a diaphragm in an accumulator of the implantable drug delivery device. An electronics module 32 such as described above may detect the displacement (i.e., the amount of deflection) of the diaphragm as a function of time.

In block 802, the electronics module 32 may begin the flow rate measurement. In an embodiment, the electronics module 32 may begin the flow rate measurement at a pre-determined time or may begin the measurement in response to a command that is received from an external device 34, such as an external programmer.

In block 804, the electronics module 32 may detect the position or deflection of the diaphragm, P₁, at a first time, T₁. For example, the electronics module 32 may detect the position (i.e., the deflection) of the diaphragm when the accumulator 30 is in a filled state, such as shown in FIG. 2C, where the diaphragm 40 is in a maximum (e.g., upwardly) deflected position. The initial time, T₁, may correspond to the time at which the outlet valve 28 of the accumulator 30 is opened and the infusate begins to empty from the accumulator (see FIG. 2D). Thus, in some embodiments the electronics module 32 may synchronize the detection of the diaphragm position P₁ with the opening of outlet valve 28. Alternately, in some embodiments the electronics module 32 may detect the position P₁ of the diaphragm 40 at any arbitrary time during the fill/empty cycle of the accumulator 30.

The electronics module 32 may detect the position or deflection of the diaphragm using diaphragm sensor data from a diaphragm sensor device configured to determine the position (i.e., the amount of deflection) of the diaphragm within the accumulator, such as any of the diaphragm sensors 302, 402, 502, 602 and/or 702 described above with reference to FIGS. 3-7.

In block 806, the electronics module 32 may detect the position or deflection of the diaphragm P₂, at a second time, T₂. The second time T₂ may be later than the first time T₁ by a known or measurement time period (i.e., ΔT). The time period may be less than about 5 seconds, such as less than about 1 second, including less than about a half-second, less than about a quarter second, less than about one-hundredth of a second, less than about a millisecond, etc. The electronics module 32 may detect the position or deflection of the diaphragm, P₂, using diaphragm sensor data from a diaphragm sensor device configured to determine the position (i.e., the amount of deflection) of the diaphragm within the accumulator, such as any of the diaphragm sensors 302, 402, 502, 602 and/or 702 described above with reference to FIGS. 3-7.

The electronics module 32 may determine the change in position or deflection of the diaphragm (i.e., the difference between P₁ and P₂, or ΔP) over the measurement time period, ΔT. As discussed above, the change in position or deflection of the diaphragm as a function of time may be directly related to the rate at which the infusate is pumped from the accumulator. In some embodiments, the electronics module 32 may determine how much the diaphragm moves (i.e., deflects) over a predetermined time period, ΔT. In other embodiments, the electronics module 32 may regularly or continuously monitor the position or deflection of the diaphragm until the diaphragm moves (i.e., deflects) by a pre-determined amount (i.e., ΔP), and may then determine the amount of time elapsed (i.e., ΔT) during the pre-determined change in diaphragm position. For example, the electronics module 32 may be configured to determine the time it takes for the diaphragm to move between an initial upwardly-deflected position P₁ in which the accumulator 30 is in a filled state, as shown in FIG. 2C, to a second position, P₂, in which the diaphragm 40 is fully deflected downwards as shown in FIG. 2A.

In determination block 808, the processor 43 of the electronics module 32 may determine whether the detected change in position or deflection of the diaphragm over the measurement time period (i.e., ΔP/ΔT) satisfies one or more threshold criteria. The at least one threshold criteria may be related to the flow rate of the infusate during normal operation of the implantable drug delivery device. In other words, the detected change in position or deflection of the diaphragm over the measurement time period (i.e., ΔP/ΔT) may be compared to a stored value corresponding to the expected change in position or deflection of the diaphragm over the same time period for a normally-operating device. The detected ΔP/ΔT may satisfy the one or more threshold criteria when the detected ΔP/ΔT deviates from the expected ΔP/ΔT by less than a predetermined amount (e.g., 0-10%). For example, if the detected ΔP/ΔT is less than a first stored threshold value, this may indicate that there is a blockage or occlusion in the flow path of the implantable drug delivery device, and that the flow rate of the device is abnormal. In another example, if the detected ΔP/ΔT is greater than a second stored threshold value (which may be the same or greater than the first threshold value), this may indicate that there is a leak or other problem in the device.

In some embodiments, the processor 43 of the electronics module may optionally determine a flow rate of the accumulator 30 based on the detected change in position or deflection of the diaphragm over the measurement time period (i.e., ΔP/ΔT). For a fixed volume accumulator, a constant volume of infusate is dispensed each time the diaphragm 40 moves from a fully upwardly-deflected position, as shown in FIG. 2C, to a fully-downwardly deflected position, as shown in FIG. 2A. Thus, the change in position or deflection of the diaphragm, ΔP, may be equivalent to a volume, which may be expressed in mL of infusate, for example. Therefore, the detected ΔP/ΔT may be expressed as a flow rate (e.g., mL/sec.), which may be compared to one or more threshold criteria comprising predetermined flow rate value(s) corresponding to normal and/or abnormal flow rates of the implantable drug delivery device.

In response to determining that the detected change in position or deflection of the diaphragm over the measurement time period (i.e., ΔP/ΔT) does not satisfy one or more threshold conditions (i.e., determination block 808=“No”), the processor 43 of the electronics module 32 may determine that the flow rate of infusate is abnormal in block 810. In some embodiments, the determination of an abnormal flow rate may be the result of an occlusion or leak in the implantable drug delivery device. The processor 43 of the electronics module 32 may provide a notification of the abnormal flow rate in block 814. For example, the processor 43 may send a message to an external device 34, such an external programmer, over a wireless interface indicating that the implantable drug delivery device has an abnormal flow rate. The processor 43 may optionally take other remedial action in response to a determination of an abnormal flow rate, such as adjusting the cycling rate of accumulator and/or shutting down the system.

In response to determining that the detected change in position or deflection of the diaphragm over the measurement time period (i.e., ΔP/ΔT) satisfies the one or more threshold conditions (i.e., determination block 808=“Yes”), the processor 43 of the electronics module 32 may determine that the flow rate of infusate is normal in block 810.

In an alternative embodiment, the processor 43 within the implantable drug delivery device may be configured with processor-executable instructions to perform the operations of blocks 804 and 806 and communicate the detected diaphragm position and time values to an external device 34. In this embodiment, the processor 47 of the external programmer 34 may receive the detected values from the implantable drug delivery device and determine whether the flow rate of infusate is normal or abnormal based on a determination of whether the detected change in position or deflection of the diaphragm over the measurement time period (i.e., ΔP/ΔT) satisfies one or more threshold conditions.

As previously discussed, the efficacy of a treatment regimen carried out by the implantable drug delivery device 300 may be influenced by various impediments (e.g., blockages, occlusions, etc.) within a flow path of the implantable drug delivery device 300. However, other factors may additionally or alternatively influence the treatment regimen carried out by the implantable drug delivery device 300.

For example, the functionality or efficiency of one or more of the components of the implantable drug delivery device 300, such as the filter 24, the bellows 16, the accumulator 30, the access port 31, the electronics 32, and the catheter 36, may change over time. However, a change in functionality or efficiency may not be significant enough to immediately influence the flow rate and/or the efficacy of the treatment regimen perceived by the patient. Therefore, it may be beneficial to directly or indirectly monitor parameters associated with the functionality of one or more of the components of the implantable drug delivery device 300.

In addition, treatment regimens may include a predetermined dose administration schedule that is defined by a clinician. An amount of infusate prescribed for use during the treatment regimen may be the same amount of infusate used during the treatment regimen. In some embodiments and treatments, a treatment regimen may allow for a predetermined number of patient-initiated doses. Thus, the amount of infusate administered during the treatment regimen may change depending upon whether the patient chooses to use one or more of the allowed number of patient-initiated doses during the treatment regimen. This may result in varying lengths of time that the overall treatment regimen may be performed before the implantable drug delivery device 300 is refilled. Therefore, it may be beneficial to directly or indirectly measure information associated with an amount of infusate administered during the treatment regimen in order to more accurately predict when an infusate refill may be needed.

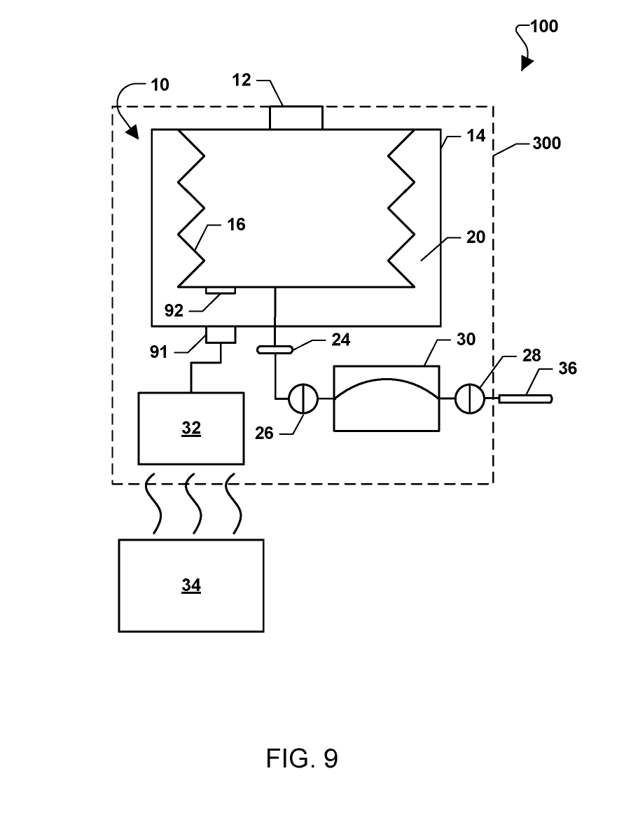

In various embodiments, methods and systems for directly or indirectly measuring or determining an amount of infusate and/or a flow rate of infusate provided to a patient may be performed by measuring various parameters associated with the drug reservoir 10. As illustrated in FIGS. 9-15, various embodiments may include an implantable drug delivery device 300 that includes a bellows sensor for detecting various parameters associated with the drug reservoir. For example, the bellows sensor may measure one or more parameters associated with the bellows 16 and/or the second zone 20 of the drug reservoir 10 that are related to the volume of infusate within the drug reservoir. In some embodiments, the bellows sensor may be configured to detect one or more of a position or displacement, pressure, or a fluid level/amount of the infusate within the bellows 16 or the two-phase liquid in the second zone 20 of the drug reservoir 10. While various bellows sensors are described with reference to FIGS. 9-15, the bellows sensor(s) may be any suitable sensor that is configured to provide information associated with one or more parameters corresponding to or associated with the infusate volume, the bellows 16, the second zone 20, and/or the two-phase liquid in order to determine an amount of infusate and/or a flow rate of infusate provided to the patient. In addition, while a single bellows sensor is illustrated in FIGS. 9-15, any number and any combination of different bellows sensors may be utilized to detect the one or more parameters associated with the bellows 16 and/or the second zone 20 of the drug reservoir 10.

Referring to FIG. 9, an implantable drug delivery device 300 may include an electronically-based bellows sensor 92 configured to communicate information associated with a position or expansion/compression state of the bellows 16. The bellows sensor 92 may include one or more electronically-based bellows sensors. While the bellows sensor 92 is illustrated in FIG. 9 as being disposed on an outer surface of the bellows 16 within the second zone 20, the bellows sensor 92 may alternatively or additionally be disposed on another portion of the outer surface of the bellows 16 or on one or more inner surfaces of the bellows 16. In some embodiments, the bellows sensor 92 may be an integral part of the bellows 16, while in other embodiments the bellows sensor 92 may be a separate device coupled to the inner or outer surface.

In some embodiments, the bellows sensor 92 may be configured to convert mechanical properties of the bellows 16 into an electrical signal that is conveyed to the electrical signal detector 91. The bellows sensor 92 may be configured or calibrated to provide information corresponding to or indicative of a displacement and/or expansion of the bellows 16. For example, the bellows sensor 92 may be configured to emit different electrical signals in response to the bellows 16 being in different displacement and/or expansion/compression states. For example, the bellows sensor 92 may generate a range electrical signals (e.g., analog or digital signals) corresponding to or indicative of the range of displacement and/or expansion/compression of the bellows 16 between full (i.e., containing the maximum amount of infusate) and empty (i.e., containing the minimum amount of infusate). As another example, the bellows sensor 92 may emit a first electrical signal when the bellows 16 is substantially empty, a second electrical signal when the bellows 16 is filled with a maximum amount of infusate (e g, immediately following a refill), a third electrical signal when a minimum amount of infusate remains within the bellows 16 (e.g., after the final dose of the treatment regimen is administered), and a fourth electrical signal when an amount of infusate within the bellows 16 is less than the maximum amount and greater than the minimum amount of infusate remains within the bellows 16.

The electrical signal detector 91 may be configured to detect the electrical signals emitted by the bellows sensor 92. The electrical signal detector 91 may be coupled to the electronics module 32. The electrical signal detector 91 may be integral with the electronics module 32 or the electrical signal detector 91 may be separate from and/or spaced away from the electronics module 32.

In some embodiments, the electrical signal detector 91 may be configured to send the detected electrical signals emitted by the bellows sensor 92 to the electronics module 32. In some embodiments, the electrical signal detector 91 may be configured to monitor or analyze electrical signals from the bellows sensor 92 and send information to the electronics module 32 in response to determining that there is a change or a rate of change in the electrical signals from the bellows sensor 92 that exceeds a predetermined threshold. In some embodiments, the electrical signal detector 91 or the bellows sensor 92 may include a processor configured to determine whether a change or rate of change in electrical signals from the bellows sensor 92 exceeds a predetermined threshold. In some embodiments, the electrical signal detector 91 or the bellows sensor 92 may be configure to compare the change or rate of change in electrical signals from the bellows sensor 92 to a plurality of predetermined thresholds. For example, a first threshold may be related to a prescribed flow rate of infusate to the patient (e.g., depending upon the dosage regimen configured by a physician), a second predetermined threshold may be related to scheduling a refill of the implantable drug delivery device 300 (e.g., a threshold that indicates infusate usage exceeds the amount assumed when a refill date was initially scheduled), and a third predetermined threshold may be indicative of a problem with the implantable drug delivery device 300, and a fourth predetermine threshold may correspond to an unsafe flow rate of infusate (e.g., triggering a warning or alarm).

In some embodiments, the bellows sensor 92 may include one or more strain gauges that generate an electrical signal by varying an electrical characteristic (e.g., resistance) in response to changes in shape caused by expansion or contraction of the bellows 16. A strain gauge bellows sensor 92 may be configured and attached to or formed within the bellows 16 so that the amount of change in the electrical characteristic due to expansion and contraction of the bellows corresponds to an amount of infusate within the bellows 16 and/or the gas to liquid ratio of the two-phase liquid within the second zone 20. Non-limiting examples of strain gauges that may be used in the bellows sensor 92 include bonded foil strain gauges, bonded semiconductor strain gauges (e.g., a piezoresistor), thin film strain gauges (e.g., a strain gauge formed by vapor deposition or sputtering of an insulator and gauge material onto a surface of the bellows or diaphragm), diffused or implanted semiconductor strain gauges, and combinations thereof.

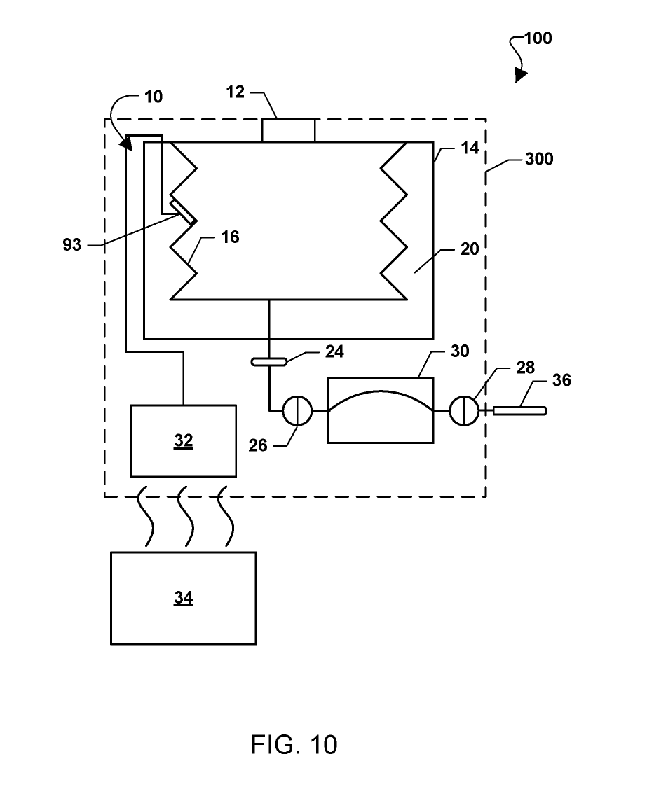

Referring to FIG. 10, an implantable drug delivery device 300 may include a bellows sensor 93 configured to communicate information associated with a position, orientation, expansion/compression state, and/or pressure associated with the bellows 16 to the electronics module 32. The bellows sensor 93 may be any sensor configured to detect a position, angle, and/or displacement associated with the bellows 16 and/or one or more leaves of the bellows 16. For example, the bellows sensor 93 may include one or more of a flex sensor, a gyroscope, a tilt sensor, a piezoelectric sensor, a linear, angular, and/or multi-axis position sensor, or a combination thereof. Additionally or alternatively, the bellows sensor 93 may be configured to measure information associated with a fluid level of the infusate within the bellows 16.

As illustrated in FIG. 10, the bellows sensor 93 may be disposed within one or more leaves of the bellows 16 and/or disposed on another outer surface of the bellows 16. As the implantable drug delivery device 300 may operate in any orientation (e.g., the patient may be standing up, lying down, etc.), the orientation information may be used by the electronics module 32 in conjunction with one or more other parameters for various determinations. For example, the orientation information may be used in conjunction with the detected information associated with the position, expansion/compression state, and/or pressure associated with the bellows 16 and/or the fluid level of the infusate. Additionally or alternatively, the orientation information may be used in conjunction with information detected to determine the flow rate at which the infusate is pumped from the accumulator as described in FIGS. 1-8.

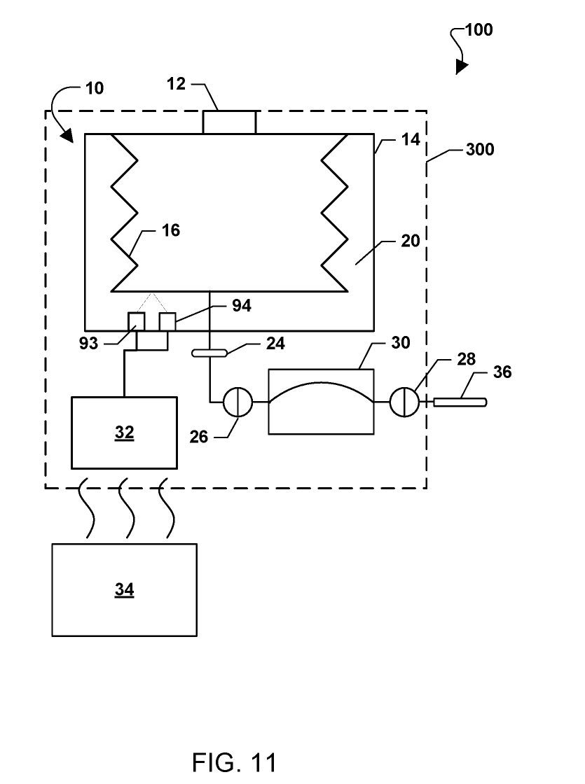

Referring to FIG. 11, an implantable drug delivery device 300 may include an optical or light-based bellows sensor including a light emitter 93 and a light detector 94. The light emitter 93 may be configured to emit one or more types of light (e.g., visible light, infrared, laser, ultraviolet, etc.) onto a surface of the bellows 16. In some embodiments, the surface of the bellows 16 in which the light emissions are directed may include a reflective material to enhance or modify the reflection of the light generated by the light emitter 93 to the light detector 94. The light generated by the light emitter 93 may reflect off of the surface of the bellows 16 and be detected and/or measured by the light detector 94. Based on a characteristic of the light (e.g., intensity, interference pattern, delay, polarity, etc.) detected/measured by the light detector 94, a position and/or expansion/compression state of the bellows 16 may be determined.

While FIG. 11 shows a light emitter 93 positioned to illuminate a bottom surface of the bellows 16, one or more pairs of light emitters 93 and light detectors 94 may be arranged to take optical measurements of one or more other surfaces of the bellows 16 (e.g., top, side, leaves, etc.).

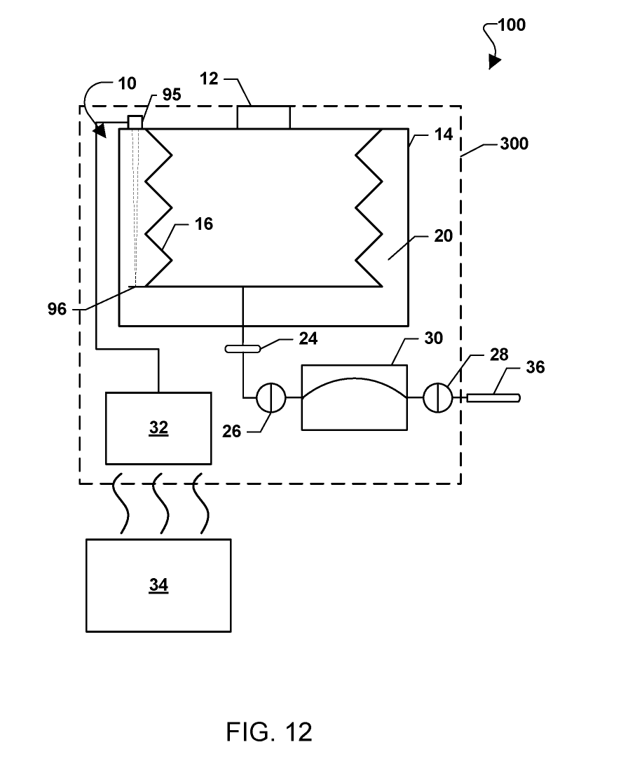

Referring to FIG. 12, an implantable drug delivery device 300 may include a light-based bellows sensor featuring a light emitter/detector 95 and an extension 96. The light emitter/detector 95 may be configured to both emit and detect one or more types of light (e.g., visible light, infrared, laser, ultraviolet, etc.) through a second zone 20 and onto a surface of the extension 92, which may be an attachment that extends from a surface of the bellows 16. The extension 96 may be made of a material having a reflective property or include a reflective material provided on a surface of the extension 96. Light emitted by the light emitter/detector 95 may reflect off of the surface of the extension 96 and be detected or measured by the light emitter/detector 95. Based on a characteristic of the light detected or measured by the light emitter/detector 95, a position and/or expansion/compression state of the bellows 16 may be determined.

While FIG. 12 shows one light emitter/detector 95 arranged outside the second zone 20, one or more light emitter/detectors 95 may be arranged with respect to any extension from any surface of the bellows 16 (e.g., top, side, leaves, etc.).

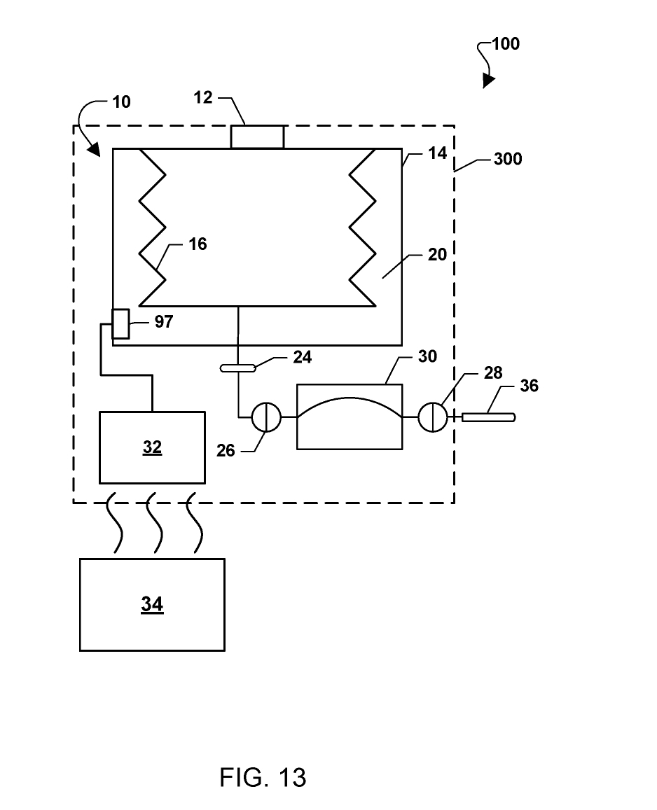

Referring to FIG. 13, an implantable drug delivery device 300 may include a bellows sensor 97 configured to detect the presence of vapor and/or liquid within the two-phase liquid within the second zone 20. For example, as previously discussed, the ratio of an amount of the two-phase liquid in a vapor state to an amount of the two-phase liquid in a liquid state may be indicative of an amount of pressure needed to pressurize the bellows 16 to perform the treatment regimen. For example, as the amount of infusate within the bellows 16 decreases, the amount of pressure provided to an outer surface of the bellows 16 by the two-phase liquid may increase in order to maintain stasis. Therefore, the bellows sensor 97 may detect an amount of the two-phase liquid in the vapor state and/or an amount of the two-phase liquid in the liquid state in order to determine an amount of infusate remaining within the bellows 16.

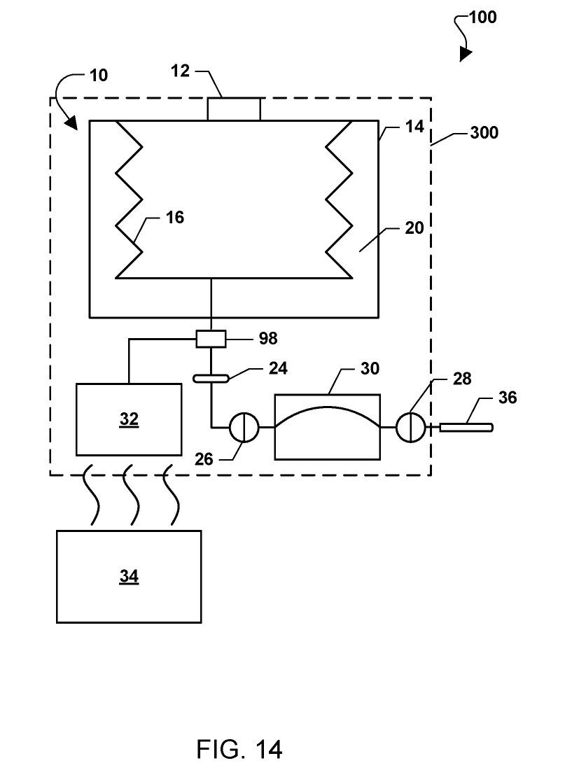

Referring to FIG. 14, an implantable drug delivery device 300 may include a flow meter 98 configured to detect a flow of infusate as it travels between the drug reservoir 10 and the accumulator 30. While the flow meter 98 is illustrated in FIG. 14 as being disposed between the housing 14 and the filter 24, one or more flow meters 98 may be disposed between the filter 24 and the valve 26 and configured to measure an amount of infusate and/or a rate at which the infusate flows between the drug reservoir 10 and the accumulator 30. The flow meter 98 may provide information associated with the amount of infusate and/or the rate at which the infusate flows between the drug reservoir 10 and the accumulator 30 to the electrics module 32.

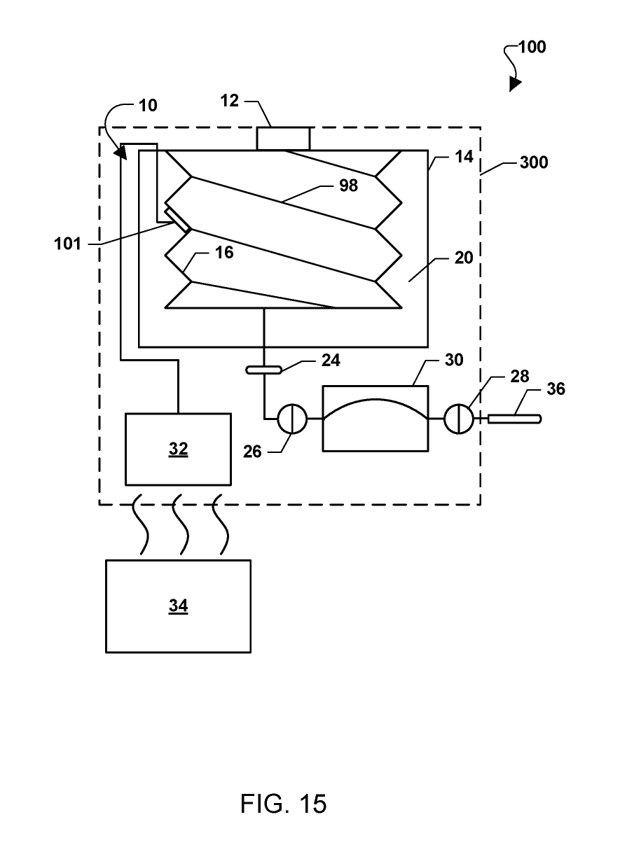

Referring to FIG. 15, an implantable drug delivery device 300 may include a coil 99 wrapped around an outer surface of the bellows 16 and an inductive bellows sensor 101. In some embodiments, the coil 99 may be disposed within the leaves of the bellows 16 as illustrated in FIG. 15. In other embodiments, the inductive bellows sensor 101 may be arranged in any position that enables measurement of at least a portion of the magnetic field generated by the coil 99. In some embodiments, the magnetic field measurements and/or the change in the magnetic field associated with the coil 99 detected by the inductive bellows sensor 101 may be used to determine an amount of infusate within the bellows 16. For example, as the amount of infusate within the bellows 16 decreases (e.g., as infusate is administered during the of the treatment regimen), the loops of the coil 99 may move closer together as the leaves of the bellows 16 compress, which may cause a change in the magnetic field generated by the coil 99. Likewise, as the bellows 16 is refilled with infusate and the leaves of the bellows 16 expand, the loops of the coil 99 may move further away from each other, changing the magnetic field in the opposite manner.

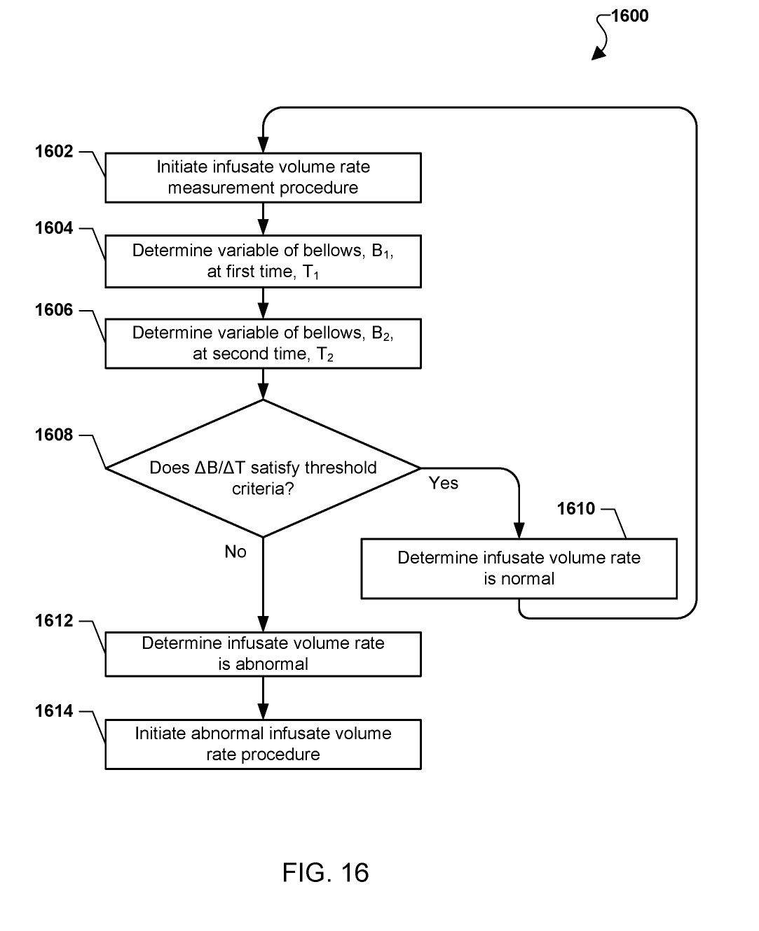

FIG. 16 is a process flow diagram illustrating an embodiment method 1600 for determining whether a change in the volume of infusate within a bellows (e.g., 16) of an implantable drug delivery device is normal. With reference to FIGS. 1-16, the method 1600 may be implemented by one or more processors of an implantable drug delivery device, a patient programmer, or a combination thereof. For example, the method 1600 may be implemented by a processor 43 of the electronics module 32 and/or a processor 47 of the external programmer 34.

In block 1602, the processor may initiate an infusate volume change rate measurement procedure. The infusate volume change rate measurement procedure may be configured to directly or indirectly determine a rate of change in volume of infusate within the bellows. In addition, the infusate volume change rate measurement procedure may use the determined rate of change in volume of the infusate within the bellows to determine a number factors, such as an efficacy of a treatment regimen, or a change or rate of change in a functionality of the implantable drug delivery device or components thereof (e.g., detect wear out or failure of a component).

The infusate volume change rate measurement procedure may be initiated by the processor in various ways. In some embodiments, the infusate volume change rate measurement procedure may be initiated by the processor in response to communication between the external programmer 34 and the electronics modules 32 of the implantable drug delivery device 300. For example, the processor 47 of the external programmer 34 may generate and transmit a message to the electronics module 32 including an instruction to initiate the infusate volume rate measurement procedure. The instruction to initiate the infusate volume change rate measurement procedure may be initiated by the external programmer 34 or by the electronics module 32 in response to communications with the external programmer 34.

In some embodiments, the infusate volume change rate measurement procedure may be triggered by a predetermined event or operation, such as filling or refilling of the bellows 16 with infusate, receiving a treatment regimen, receiving an indication to modify one or more parameters of the currently implemented treatment regimen, determining that the flow rate is abnormal, etc.

Additionally or alternatively, the volume rate measurement procedure may be initiated by the processor at periodic intervals during a treatment regimen. The number of times that an infusate volume change rate measurement is performed during a treatment regimen and/or a time interval between infusate volume change rate measurements may be predetermined or dynamically determined by the processor for a current treatment regimen, for a predetermined number of sequential treatment regimens, and/or for the anticipated life of the implantable drug delivery device. For example, information associated with the number of times a volume change rate measurement is performed and/or the time interval between volume change rate measurements may be included in one or more of the information associated with the current treatment regimen or information associated with the operation of the implantable drug delivery device provided at manufacture, prior to implantation, and/or after implantation. The information associated with the number of times the volume change rate measurement is performed and/or the time interval between volume change rate measurements may be predefined times or intervals, or information that may allow the processor to determine the number of times the volume change rate measurement is performed and/or the time interval between volume change rate measurements.

The number of performances and/or the time interval between volume change rate measurements may vary over time. The volume change rate measurements may be performed by the processor more or less frequently over a time interval associated with one treatment regimen, a plurality of sequential treatment regimens, and/or a lifetime of the implantable drug delivery device. In some embodiments, the volume change rate measurements may be performed by the processor more frequently immediately following filling or refilling of the bellows in order to determine an initial maximum volume of infusate for the current treatment regimen. Alternatively, the volume change rate measurements may be performed by the processor less frequently following filling or refilling of the bellows 16.

In some embodiments, the processor may perform volume rate measurements more frequently toward the end of the current treatment regimen. For example, in order to prevent an interruption in the delivery of infusate to the patient, it may be desirable to more closely monitor the volume of the infusate such that measurements may be taken to prevent the implantable drug delivery device from emptying the bellows of infusate prior to refilling the bellows 16 with more infusate. In some embodiments, in response to determining that the current volume of the infusate is less than a predetermined threshold but greater than a minimum infusate volume level, the rate at which the volume rate measurement is performed may be increased by the processor to more granularly monitor the consumption of infusate. An alert may be transmitted by the processor to the patient and/or clinician in response to determining that the current volume of the infusate is less than the predetermined threshold value. The alert associated with the infusate level may be provided as an indication or reminder that a refill of infusate is needed within a predetermined amount of time.

In block 1604, the processor may determine a first variable of the bellows (B₁) at a first time (T₁). The first variable B₁ may be associated with a volume of infusate within the bellows 16 and may be detected or determined using an output of one or more bellows sensors and/or detectors associated with the bellows 16. For example, the electrical signal detector 91, bellows sensor 93, light detector 94, light emitter/detector 95, bellows sensor 97, flow meter 98, and/or inductive bellows sensor 101 may be electrically coupled to the processor such that the processor receives an output signal that corresponds to a measured parameter associated with the infusate and/or the bellows 16. In some embodiments, the output of the one or more bellows sensors and/or detectors associated with the bellows 16 may correspond to a volume value of infusate within the bellows 16. In other embodiments, the processor may use the first variable B₁ to calculate the volume of infusate within the bellows 16.

The processor may store the determined first variable of the bellows B₁. In addition, the processor may store information associated with the first time T₁ in a memory. In some embodiments, the first time T₁ may be a time stamp or other value associated with a discrete time instance that corresponds to when the parameter of the bellows 16 is measured. In other embodiments, the processor may initiate a timer in response to receiving the output corresponding to the measured parameter of the bellows 16 used to determine the first variable of the bellows B₁.

In block 1606, the processor may determine a second variable of the bellows (B₂) at a second time (T₂). The second variable B₂ may be associated with a volume of infusate within the bellows 16 and may be detected or determined by the processor using an output of one or more bellows sensors and/or detectors associated with the bellows 16. For example, the electrical signal detector 91, bellows sensor 93, light detector 94, light emitter/detector 95, bellows sensor 97, flow meter 98, and/or inductive bellows sensor 101 may be configured to generate an output to the processor that corresponds to a measured parameter of the bellows 16. In some embodiments, the output of the one or more bellows sensors and/or detectors associated with the bellows 16 may correspond to a volume value of infusate within the bellows 16. In other embodiments, the processor may use the second variable B₂ to calculate the volume of infusate within the bellows 16.

The processor may store the determined second variable of the bellows B₂. In addition, the processor may store information associated with the second time T₂ in a memory. In some embodiments, the second time T₂ may be a time stamp or other value associated with a discrete time instance that corresponds to when the parameter of the bellows 16 is measured. In other embodiments, the processor may stop the initiated timer in response to receiving the output corresponding to the measured parameter of the bellows 16 used to determine the second variable of the bellows B₂.

In determination block 1608, the processor may determine whether a change between the first variable of the bellows B₁ and the second variable of the bellows B₂ over the time period between the first time T₁ and the second time T₂ satisfies a threshold criterion. The processor may implement any of a variety of calculations for determining a rate of change in volume of infusate within the bellows 16 based on two measured parameters. In an exemplary embodiment, the following equation may be used to determine the rate of change between the first variable of the bellows B₁ and the second variable of the bellows B₂ over the time period between the first time T₁ and the second time T₂:

$$\frac{Δ B}{Δ T} = \frac{\vert {B}_{1} - {B}_{2} \vert}{{T}_{2} - {T}_{1}}$$

After the rate of change ΔB/ΔT is determined, the processor may compare the resulting value to a threshold criterion. In some embodiments, the threshold criterion may be predetermined, while in other embodiments the threshold criterion may be dynamically determined by the processor based on parameters associated with one or more of the current treatment regimen, a predetermined number of sequential treatment regimens, and/or for an anticipated life of the implantable drug delivery device. The threshold criterion may be static for a single treatment regimen or the threshold criteria may be dynamically determined by the processor in response to determining that the infusate volume may be abnormal. The threshold criterion may be a single value or a range of values.

In response to determining that the rate of change ΔB/ΔT satisfies the threshold criterion (i.e., determination block 1608=“Yes”), the processor may determine that the infusate volume change rate is normal in block 1610.

In response to determining that the rate of change ΔB/ΔT does not satisfy the threshold criterion (i.e., determination block 1608=“No”), the processor may determine that the infusate volume change rate is abnormal in block 1610. In some embodiments, a determination that the infusate volume change rate is abnormal may indicate that the determined volume of infusate in the bellows does not match an amount or volume of anticipated infusate remaining. A difference between a determined volume and an anticipated volume of infusate may be caused by various factors, such as an intentional override of a prescribed dose by the patient, an additional dose administered to the patient, a change in function of one or more elements of the implantable drug delivery device, etc.

In some embodiments, the processor may determine that the infusate within the bellows does not satisfy the threshold criterion in response to determining that the infusate within the bellows exceeds the threshold criterion. For example, a situation in which the flow path between the bellows and the catheter is unimpeded (e.g., an open valve) may be detected when the processor determines that the infusate within the bellows is less than a threshold criterion (i.e., too much infusate has been drained from the bellows). As another example, a situation in which the flow path between the bellows and the catheter is impeded, such as from a catheter occlusion, may be detected when the processor determines that the infusate within the bellows is greater than a threshold criterion (i.e., too little infusate has been administered to the patient under a prescribed flowrate). In some embodiments, the threshold criterion may be a rate of change in volume within the bellows, in which case an unimpeded flow path may be detected when the volume change rate exceeds a threshold criterion and an impeded flow path may be detected when the processor determines that the volume change rate is less than a threshold criterion. For ease of reference, such determinations by the processor may be referred to as determining whether a measurement satisfies a threshold criterion.

In block 1614, the processor may initiate an abnormal infusate volume change rate procedure in response to determining that the infusate volume rate is abnormal. The abnormal infusate volume rate procedure performed by the processor may include various operations configured to modify the current infusate volume change rate measurement procedure, notify a patient and/or clinician that an abnormal operating state is detected, determine a potential cause triggering the abnormal operating state, and/or discontinue operation of the implantable drug delivery device.

In some embodiments, a plurality of predetermined operations may be stored in memory and in response to determining a potential cause for the abnormality and/or a level of abnormality, the processor may retrieve and execute the stored operation that corresponds to the determined potential cause. In other embodiments, the operations may be dynamically selected the infusate volume rate is abnormal to create a unique abnormal infusate volume rate procedure based on current measurements.

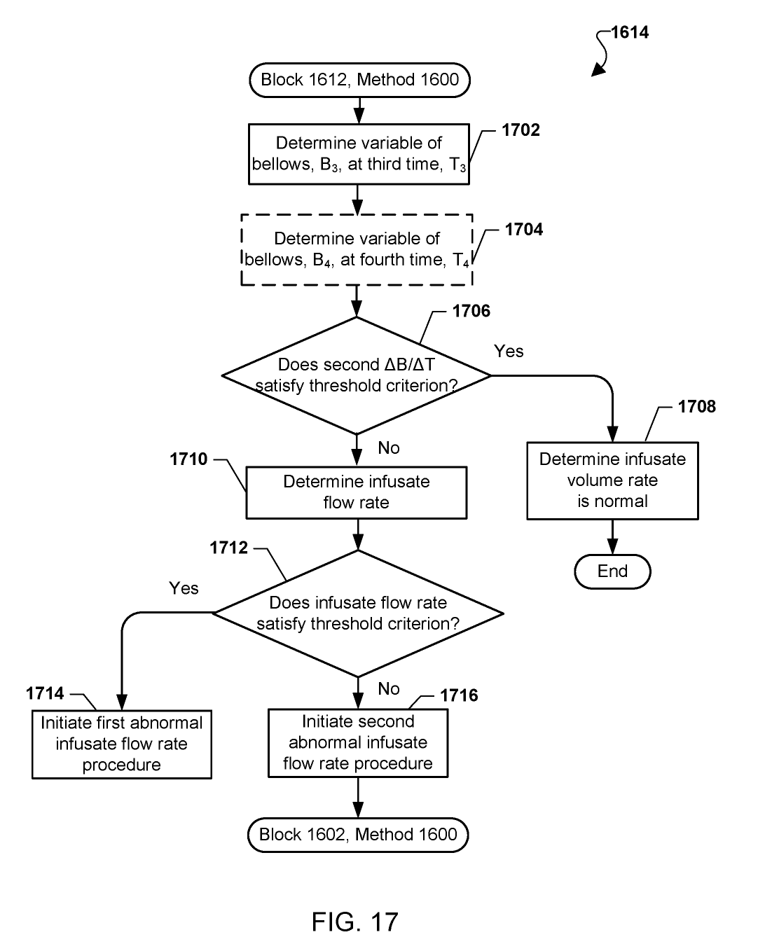

FIG. 17 is a process flow diagram of a non-limiting example of an abnormal infusate volume change rate procedure 1614. With reference to FIGS. 1-17, the method 1614 may be implemented by one or more processors (e.g., processor 43 and/or processor 47) of an implantable drug delivery device (e.g., implantable drug delivery device 100), a patient programmer (e.g., external programmer 34), or a combination thereof.

In block 1702, the processor may determine a third variable of the bellows (B₃) at a third time (T₃) in response to determining that the infusate volume change rate is abnormal in block 1612. The third variable B₃ may be associated with a volume of infusate within the bellows 16 and may be detected or determined using an output of one or more bellows sensors and/or detectors associated with the bellows 16. The processor may store the determined third variable B₃ as well as information associated with the third time T₃.

In some embodiments, determining the third variable of the bellows B₃ may be performed to confirm the validity of the abnormal determination. Various factors may inadvertently influence the parameter of the bellows 16 measured by the bellows sensor and/or detector at a discrete time instance (e.g., T₁ or T₂) without influencing the actual infusate volume within the bellows 16. For example, depending on the current state of the pumping cycle and/or the cycling rate of the accumulator, the infusate may be displaced within the bellows 16 such that any measurement associated with a height level and/or surface of the infusate within the bellows 16 may be inaccurate.

In optional block 1704, the processor may determine a fourth variable of the bellows (B₄) at a fourth time (T₄). The fourth variable B₄ may be associated with a volume of infusate within the bellows 16 and may be detected or determined by the processor using an output of one or more bellows sensors and/or detectors associated with the bellows 16. The processor may store the determined fourth variable B₄ as well as information associated with the fourth time T₄.

In determination block 1706, the processor may determine whether a change between at least two of the first variable of the bellows B₁, the second variable of the bellows B₂, the third variable of the bellows B₃, and the fourth variable of the bellows B₄ over a time period satisfies a threshold criterion. The processor may implement any of a variety of calculations for determining a rate of change in volume of infusate within the bellows 16 based on two or more measured parameters.

The processor may determine an amount of change between two or more of the determined variables of the bellows based on the measured parameters. For example, the processor may determine an amount of change between one or more of the third variable of the bellows B₃ and the fourth variable of the bellows B₄ (ΔB₃₄), the first variable of the bellows B₁ and the third variable of the bellows B₃ (ΔB₁₃), the first variable of the bellows B₁ and the fourth variable of the bellows B₄ (ΔB₁₄), the second variable of the bellows B₂ and the third variable of the bellows B₃ (ΔB₂₃), and the second variable of the bellows B₂ and the fourth variable of the bellows B₄ (ΔB₂₄). The processor may determine an amount of change between the time intervals that correspond to the determined amount of change between the variables of the bellows.

After the one or more rates of change ΔB/ΔT are determined, the processor may compare the resulting values to a threshold criterion. In some embodiments, the threshold criteria may be a predetermined value, while in other embodiments, the threshold criterion may be dynamically determined by the processor based on parameters associated with one or more of the current treatment regimen, a predetermined number of sequential treatment regimens, and/or for an anticipated life of the implantable drug delivery device. The threshold criterion may be static for a single treatment regimen or the threshold criteria may be dynamically determined in response to determining that the infusate volume may be abnormal. The threshold criterion may be a single value or a range of values.

In some embodiments, the threshold criterion may be another determined rate of change during the current treatment regimen. For example, when the processor determines an amount of change between the third variable of the bellows B₃ and the fourth variable of the bellows B₄ (ΔB₃₄), the selected threshold criterion may be the amount of change between the first variable of the bellows B₁ and the second variable of the bellows B₂ (ΔB₁₂) to determine whether the infusate is reduced at a uniform rate.

Alternatively or additionally, the threshold criterion may be one or more rates of change determined during a different treatment regimen. The threshold criterion may include an amount of change between variables of the bellows determined during one or more previous treatment regimens. In some embodiments, the selected threshold criterion may correspond to a substantially similar time frame within the current and previous treatment regimen or to a substantially similar infusate volume level during the current and previous treatment regimen. In some embodiments, the selected threshold criterion may correspond to any time frame within one or more of the previous treatment regimens. For example, if an abnormality is determined, the processor may determine whether a change in functionality of one or more elements of the implantable drug delivery device has occurred based on the threshold criterion selected from one or more previous treatment regimens.

In response to determining that the rate of change ΔB/ΔT satisfies the threshold criterion (i.e., determination block 1706=“Yes”), the processor may determine that the infusate volume rate is normal in block 1708, terminate the procedure. In some embodiments, the processor may determine that the determination of an abnormal infusate volume change rate in block 1612 was a false positive based on whether the rate of change ΔB/ΔT satisfies the threshold criterion.

In response to determining that the rate of change ΔB/ΔT does not satisfy the threshold criteria (i.e., determination block 1706=“No”), the processor may determine the infusate flow rate in block 1710. The processor may determine the infusate flow rate using various techniques, including the methods described herein. For example, the processor may determine the infusate flow rate using an output of a bellows sensor and/or a detector associated with the accumulator. The processor may also determine the infusate flow rate using an output of a flow rate sensor associated with the fluid flow within the implantable drug delivery device or the catheter.

In determination block 1712, the processor may determine whether the infusate flow rate satisfies a threshold criterion. The threshold criterion may be a predetermined value or may be dynamically determined by the processor during the current treatment regimen using various parameters. In some embodiments, the threshold criterion may be based on an infusate flow rate determined in one or more previous treatment regimens.

In response to determining that the infusate flow rate satisfies the threshold criterion (i.e., determination block 1712=“Yes”), the processor may initiate a first abnormal infusate flow rate procedure, which may include various operations configured to identify one or more elements of the implantable drug delivery device that may be causing the abnormal flow rate and modify the flow rate or discontinue operation of the implantable drug delivery device. The first abnormal infusate flow rate procedure may further also include various operations to notify a patient and/or a clinician. In some embodiments, the threshold criterion value or range of values may be indicative of a change in operation of one or more elements of the implantable drug delivery device causing the abnormal flow rate.

In response to determining that the infusate flow rate does not satisfy the threshold criterion (i.e., determination block 1712=“No”), the processor may initiate a second abnormal infusate flow rate procedure that is different from the first abnormal infusate flow rate procedure. The second abnormal infusate flow rate procedure may include various operations configured to modify the current infusate volume rate measurement procedure, modify the current treatment regimen, and/or determine a potential cause triggering the abnormal flow rate. The second abnormal infusate flow rate procedure may not discontinue the operation of the implantable drug delivery device. Therefore, after the second abnormal infusate flow rate procedure is executed, the processor may continue to perform normal operations including repeating the infusate volume change rate measurement procedure of the method 1600 at an appropriate time.

The first and second abnormal infusate flow rate procedures may be predetermined and uniform for the life of the implantable drug delivery device. Alternatively, the first and second abnormal infusate flow rate procedures may be dynamically determined and/or modified by the processor. In some embodiments, the plurality of predetermined operations of the first or second abnormal infusate flow rate procedures may be stored in memory, and in response to determining a potential cause for the abnormal flow rate, the processor may retrieve and execute the stored operations that corresponds to the determined potential cause. In some embodiments, the operations may be dynamically selected by the processor to create a unique abnormal infusate flow rate procedure based on current measurements and the parameters of the current treatment regimen.

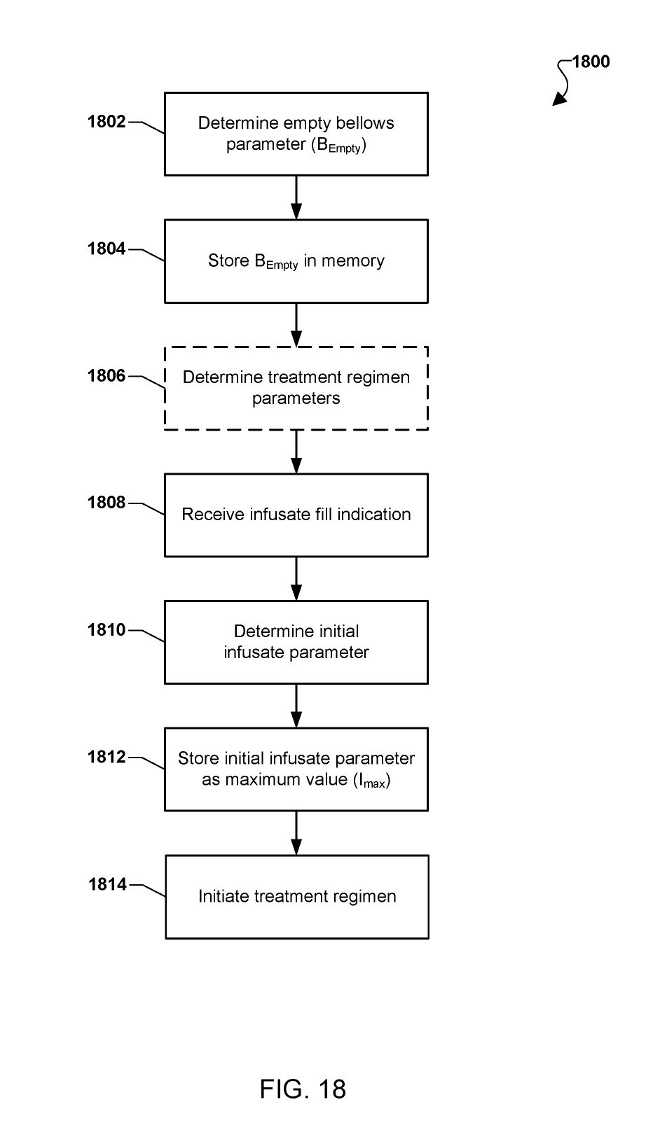

FIG. 18 is a process flow diagram of an embodiment method 1800 for determining initial parameters associated with a bellows of an implantable drug delivery device. With reference to FIGS. 1-18, the method 1800 may be implemented by one or more processors (e.g., processor 43 and/or processor 47) of an implantable drug delivery device (e.g., implantable drug delivery device 100), a patient programmer (e.g., external programmer 34), or a combination thereof.

In block 1802, the processor may determine an empty bellows parameter (B_{Empty}). The processor may determine the empty bellows parameter B_{Empty} based on one or more outputs received from one or more bellows sensors and/or detectors associated with the bellows 16 when the bellows 16 is empty or substantially empty of any infusate. The determination of the empty bellows parameter B_{Empty} may be determined once upon initialization of the implantable drug delivery device and/or each time the bellows is empty or substantially empty of any infusate.

In block 1804, the processor may store the determined empty bellows parameter B_{Empty} in a memory of the implantable drug delivery device and/or the patient programmer. In some embodiments, the empty bellows parameter B_{Empty} may be used to determine the current and/or future treatment regimens as well as any of the abnormal procedures described herein.

In optional block 1806, the processor may determine treatment regimen parameters. The treatment regimen parameters may be predefined by the clinician. Alternatively, the processor may receive information associated with the treatment regimen and use the information to determine the corresponding treatment regimen parameters. While block 1806 is illustrated after block 1804, the treatment regimen parameters may be determined at any point before, during, or after initialization of the method 1800.

In block 1808, the processor may receive an infusate fill indication. In some embodiments, the infusate fill indication may be generated by fill detector 41. In some embodiments, the processor may receive the infusate fill indication in a message from the patient programmer.

In block 1810, the processor may determine an initial infusate parameter. The initial infusate parameter may be determined based on an output of one or more of the bellows sensors and/or detectors associated with the bellows after the full dose of the infusate is stored in the bellows.

In block 1812, the processor may store the initial infusate parameter as a maximum infusate value (I_{Max}). The maximum infusate value I_{Max} may be used by the processor to determine the current and/or future treatment regimens as well as any of the abnormal procedures described herein. For example, the maximum infusate value I_{Max} may be determined by the processor after each refill of the infusate. If during the lifetime of the implantable drug delivery device, the determined maximum infusate value I_{Max} changes, such change may correspond to a change in the function of one or more of the elements of the implantable drug delivery device.

In block 1814, the processor may initiate the treatment regimen. The processor may instruct one or more elements of the implantable drug delivery device to begin administering the infusate according to the parameters of the treatment regimen.

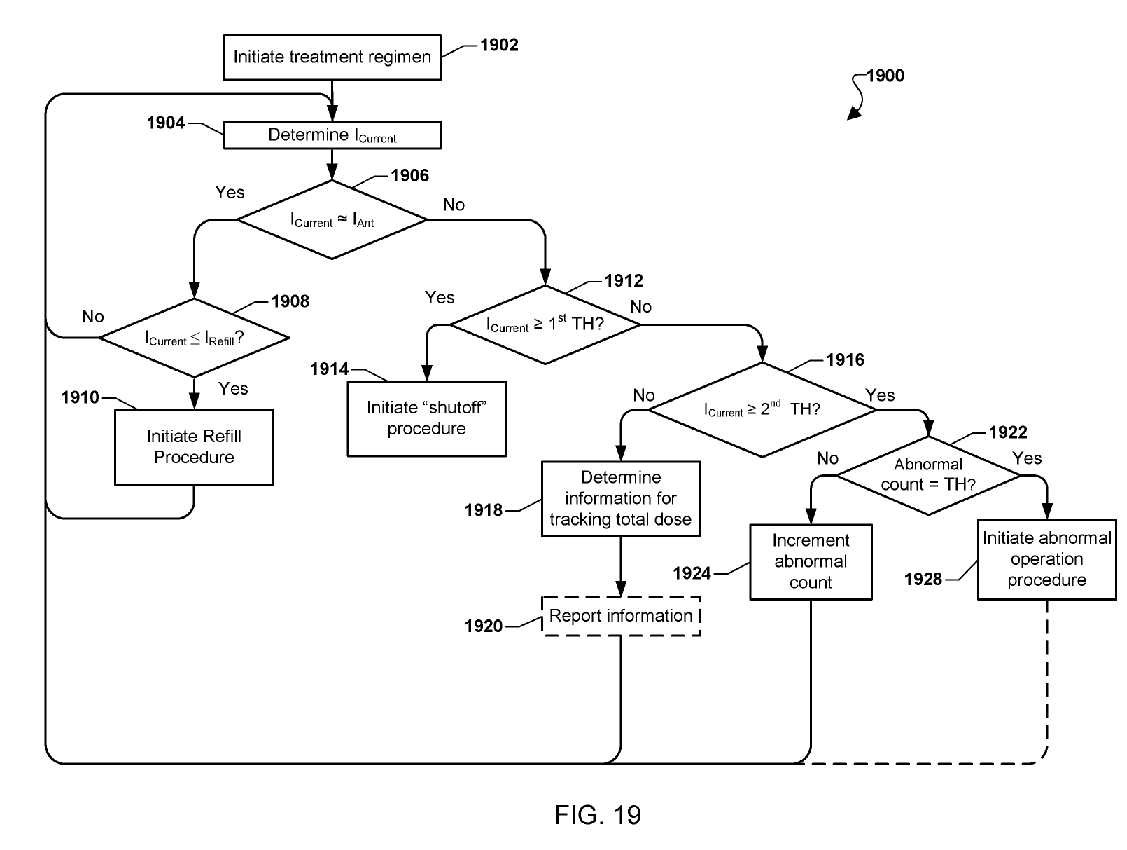

FIG. 19 is a flow diagram of an embodiment method 1900 for determining whether a change in parameter associated with an infusate in a bellows of an implantable drug delivery device is normal. With reference to FIGS. 1-19, the method 1900 may be implemented by one or more processors of an implantable drug delivery device, a patient programmer, or a combination thereof. For example, the method 1900 may be implemented by processor 43 of the electronics module 32 and/or processor 47 of the external programmer 34.

In block 1902, the processor may initiate the treatment regimen, such as by instructing one or more elements of the implantable drug delivery device to begin administering the infusate according to the parameters of the treatment regimen.

In block 1904, the processor may determine a current parameter associated with the infusate (I_{Current}) in the bellows of the implantable drug delivery device. The current parameter I_{Current} may be determined based on one or more outputs generated by one or more bellows sensors and/or detectors associated with the infusate and/or the bellows. For example, the electrical signal detector 91, bellows sensor 93, light detector 94, light emitter/detector 95, bellows sensor 97, flow meter 98, and/or inductive bellows sensor 101 may be configured to generate an output to the processor that corresponds to a measured parameter associated with the infusate and/or the bellows.

The processor may use one or more of the measured parameters determined from the outputs of the one or more bellows sensors and/or detectors associated with the infusate and/or the bellows to determine the current parameter I_{Current}. For example, the processor may use the outputs of one or more of the measured parameters to determine a current volume of infusate, a current amount of infusate, a current weight associated with the infusate, and a current height of the infusate within the bellows. When the processor uses a plurality of outputs to determine the current parameter I_{Current}, the processor may use a plurality of outputs from the same bellows sensor and/or detector or one or more outputs from different bellows sensors and/or detectors. In some embodiments, the processor may store the determined current parameter I_{Current} for additional purposes during the current treatment regimen and/or for future treatment regimens, as well as for any determinations regarding function, state, or status of the implantable drug delivery device.

In determination block 1906, the processor may determine whether the determined current parameter associated with the infusate I_{Current} is equal to or substantially equal to an anticipated parameter associated with the infusate (I_{Ant}). The anticipated parameter I_{Ant} may correspond to an amount or volume of infusate that is projected to be remaining within the bellows. The anticipated parameter I_{Ant} may be dynamically determined or selected from a predetermined value based on one or more parameters associated with the current treatment regimen, parameters associated with previous treatment regimens, parameters associated with future treatment regimens, and parameters associated with the patient. For example, some of the parameters associated with the treatment regimens may include a number of doses that may be administered using the maximum infusate amount filled in the bellows, a dosage amount, a dosage administration time interval, information associated with a time in which the last dose of infusate was administered during the current treatment regimen, a number of doses previously administered during the current treatment regimen, an amount of infusate consumed during the current treatment regimen. The processor may further consider information associated with one or more previously executed treatment regimens.

The anticipated parameter I_{Ant} may change as the treatment regimen is executed by the implantable drug delivery device. Each time a dose is administered, the anticipated parameter I_{Ant} may change such that it reflects the reduction in infusate that is consumed by the previous dose. In some embodiments, the dose of infusate may be administered at a predetermined time interval defined in the treatment regimen parameters and/or a dose may be administered in response to receiving a request from the patient. Each dose administered by the implantable drug delivery device may be uniform throughout a treatment regimen. Alternatively, the implantable drug delivery device may administer the infusate in different dosages.

In some embodiments, the different dose quantities of infusate may be based on one or more treatment regimen parameters, additional inputs or requests received from the patient, and/or parameters associated with a physical state of the patient. For example, if the implantable drug delivery device is configured to deliver insulin to a patient, a dosage amount of insulin used to produce a therapeutic effect may vary based on the parameters of the last dose administration, the hydration level of the patient, the type and/or amount of food consumed by the patient, etc.

After determining the anticipated parameter I_{Ant}, the processor may compare the determined current parameter I_{Current} in determination block 1806. In response to determining that the determined current parameter I_{Current} is equal to or substantially equal to the anticipated parameter I_{Ant} (i.e., determination block 1906=“Yes”), the processor may determine whether the determined current parameter I_{Current} is less than or equal to a desired refill value associated with the infusate (I_{Refill}) in determination block 1908. For example, the desired refill value may be greater than a minimum infusate value in order to provide the patient and/or clinician the time necessary to acquire and refill the infusate into the bellows such that a discontinuation of dose administration may be avoided.

In response to determining that the determined current parameter I_{Current} is greater than the desired refill value I_{Refill} (i.e., determination block 1908=“No”), the processor may determine the next current parameter in block 1904.

In response to determining that the determined current parameter I_{Current} is less than or equal to the desired refill value I_{Refill} (i.e., determination block 1908=“Yes”), the processor may initiate a refill procedure in block 1910, and then again determine the next current parameter in block 1904. The refill procedure may include one or more operations configured to alter or notify the patient, the clinician, and/or infusate provider that a time to refill the infusate is approaching. In some embodiments, the processor may generate a notification message that is transmitted to the external programmer 34 such that a notification may be displayed on the external programmer 34 or the external programmer 34 may further transmit the notification message to an applicable device associated with the patient, the clinician, and/or an infusate provider. Additionally or alternatively, the implantable drug delivery device may include a haptic feedback device that the processor may activate to generate s an output to the haptic feedback device to alert the patient using a vibration generated by the haptic feedback device. The refill procedure may be performed at predetermined time intervals until a refill is detected. In some embodiments, the frequency of the refill procedure may increase as the determined current parameter I_{Current} approaches a minimum amount of infusate.

In response to determining that the determined current parameter I_{Current} is not equal or substantially equal to the anticipated parameter I_{Ant} (i.e., determination block 1906=“No”), the processor may determine whether the determined current parameter I_{Current} is greater than or equal to a first threshold in determination block 1912. The first threshold may be indicative of a substantial malfunction of the implantable drug delivery device that is administering infusate at a rate exceeding the prescribed dosage, which could be injurious to the patient.

Additionally or alternatively, the processor may initiate an abnormal infusate volume procedure in response to determining that the determined current parameter I_{Current} is not equal or substantially equal to the anticipated parameter I_{Ant} (i.e., determination block 1906=“No”). For example, the processor may generate and send a message via the wireless communication interface of the implantable drug delivery device to an external device in response to determining that the determined current parameter I_{Current} is not equal or substantially equal to the anticipated parameter I_{Ant}. The message may include an indication that the current parameter I_{Current} is not equal or substantially equal to the anticipated parameter I_{Ant a} and/or the message may include the determined current parameter I_{Current}. In some embodiments, the external device may be associated with a clinician and in response to receiving information associated with the determined current parameter I_{Current}, the clinician may verify whether a volume discrepancy existed. In some embodiments, the clinician (or an external device associated with the clinician) may compare the determined current parameter I_{Current} to the prescribed flow rate multiplied by the elapsed time since the last refill event to determine whether the implantable drug delivery device requires refilling without making a physical a volume measurement by inserting a needle through the skin into the pump reservoir.

In response to determining that the current parameter I_{Current} is greater than or equal to the first threshold (i.e., determination block 1912=“Yes”), the processor may initiate a “shutoff” procedure in block 1914 in which the processor may close one or more flow control devices (e.g., valves) within the fluid path of the implantable drug delivery device to prevent an undesirable administration of the infusate to the patient.

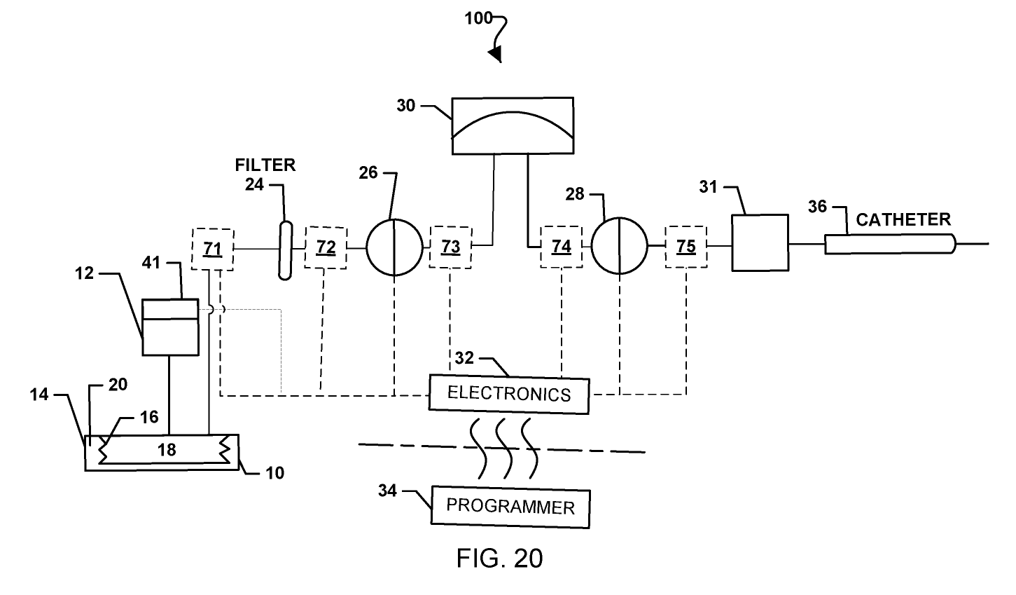

FIG. 20 is a schematic diagram of another example of an implantable drug delivery system that is configured to execute a shutoff procedure. Specifically, one or more flow control devices (e.g., valves) 71, 72, 73, 74, 75 may be arranged within the flow path between the bellows 16 and the catheter 36. During the shutoff procedure, the processor may close one or a plurality of the flow control devices 71, 72, 73, 74, and/or 75 to close. In some embodiments, the flow control device that is closed may prevent infusate from reaching the catheter.

Referring back to FIG. 19, in response to determining that the current parameter I_{Current} is less than the first threshold (i.e., determination block 1912=“No”), the processor may determine whether the current parameter I_{Current} is greater than or equal to a second threshold in determination block 1916. In some embodiments, the second threshold may indicate that the infusate usage I_{Current} exceeds the anticipated parameter I_{Ant} by an amount that is not significant enough to require immediate shutdown of the implantable drug delivery device. In some embodiments, the second threshold may indicate that the dosage administered to the patient in each treatment operation (e.g., single bolus delivery) exceeds the amount expected under the current dosage regimen by an amount that is not hazardous over a period of time. Some variation in applied dosage may occur from time to time that may be acceptable so long as the total amount or number of such events is limited. For example, in some embodiments implementing some treatment regimens, the patient may be able to initiate an increased dosage, such as by pressing a button on a patient programmer device.

In response to determining that the current parameter I_{Current} is less than the second threshold (i.e., determination block 1916=“No”), the processor may determine information relevant to or useful for assessing a total dose administered to the patient-initiated dose in block 1918. In this logic branch, the rate of infusate administered departs from the anticipated dosage rate, either high or low, but at a safe level. Departures from the anticipated dosage rate may be due to developing issues in various components or elements of the implantable drug delivery device. For example, restrictions in flow paths within the implantable drug delivery device or the catheter may reduce the amount of infusate administered over time. As another example, changes in various components over time due to wear may increase the amount of infusate administered over time. Therefore, to enable a clinician to monitor the patient treatment using the implantable drug delivery device, in block 1918 the processor may determine and record information relevant to the observed total dose for subsequent reporting to an external device (e.g., a patent programmer or a clinician programmer device). In some embodiments, such reporting may occur during a routine check of the implantable drug delivery device during which a range of operational information is reported. In some embodiments, such information may be communicated to an external device as (or soon after) it is generated in optional block 1920. The processor may continue to measure the next infusate change rate in block 1904.

In response to determining that the current parameter I_{Current} is greater than or equal to the second threshold (i.e., determination block 1916=“Yes”), the processor may determine whether a count of the number of times that an abnormal dosage measurement was detected is equal to a third threshold in determination block 1922. The third threshold may be a number of occurrences of an increased dosage that is indicative of a potential health issue for the patient if permitted to persist.

In response to determining that the count of the number of times that an abnormal dosage measurement was detected is less than the third threshold (i.e., determination block 1922=“No”), the processor may increment the count in block 1924, and return to measuring the next infusate change rate in block 1904.

In response to determining that the count of times the processor that an abnormal dosage measurement was detected equals the threshold number (i.e., determination block=“Yes”), the processor may initiate an abnormal operation procedure in block 1928. The abnormal operation procedure may involve a variety of operations to protect the patient and/or inform the clinician, and may be performed in a single abnormal operation procedure or in a plurality of different abnormal operation procedures.

For example, a first abnormal operation procedure may modify various parameters associated with the operation and/or monitoring of the operation of the implantable drug delivery device. In some embodiments, the processor may be configured to modify a procedure associated with when the one or more bellows sensors and/or detectors measure a current parameter I_{Current}. For example, the processor may reduce the time between measurements of the current parameter I_{Current}. The processor may also modify one or more of the first and second threshold criteria consistent with the abnormal determination. In some embodiments, the abnormal operation procedure may further include increasing the count threshold (i.e., the third threshold).

As another example, a second abnormal operation procedure may provide notifications to the patient and/or the clinician regarding the abnormal state of the implantable drug delivery device. Such notifications may include sending a notification to an external programmer (e.g., a patient programmer and/or a clinician programmer). In some embodiments, the implantable drug delivery device may include a haptic feedback device (e.g., a shaker) and the second abnormal operation procedure may include the processor activating the haptic feedback device to generate vibrations capable of being perceived by the patient.

As another example, a third abnormal operation procedure may include operations that the processor may perform to determine a potential cause of the abnormal delivery of infusate to the patient. The third abnormal procedure may be performed sequentially or concurrently with any other abnormal operation procedure. In some embodiments, the processor may determine whether one or more of the elements of the implantable drug delivery device is the potential cause of the abnormal operation state. Depending on the configuration of the implantable drug delivery system and the particular element(s) identified as effecting the operation of the implantable drug delivery device, the processor may initiate procedures to correct or reconfigure the identified element(s).

Referring to FIG. 20, when the element is identified as the bellows, the processor may control flow control device 71 and/or 72 such that the flow control device 71 and/or 72 may be used to regulate and meter an amount of infusate between the bellows 16 and the accumulator 30. For example, in response to identifying the bellows as the cause of the abnormal dosages of infusate, the processor implementing the third example abnormal operation procedure may close the flow control device 71 to prevent infusate from flowing between the bellows and the accumulator. Assuming that the first valve 26 was in an open position in anticipation of receiving infusate from the bellows, the processor may determine an amount of infusate that reached the accumulator before the flow control device 71 was closed. The processor may then open the flow control device 71 to allow any additional infusate to flow to the accumulator in order to achieve a full dose. Subsequently, the processor may open and close the flow control device 71 at a frequency that would allow a substantially similar amount of infusate to flow according to the current treatment regimen as if the bellows was operating normally. If both of the flow control devices 71 and 72 are implemented, the processor may further meter the flow of infusate between the flow control device 71 and the flow control device 72 such that both the flow control device 71 and the flow control device 72 are not open at the same time.

Similar procedures may be implemented for the inlet valve 26 and the outlet valve 28 as part of an abnormal operation procedure provided that an amount of infusate is still capable of flowing through each valve. For example, the processor may control the flow control device 73 to operate in a similar way in which the inlet valve 26 is configured to operate and the processor may control the flow control device 74 in a similar way in which the outlet valve 26 operates.

Alternatively, if the accumulator 30 and/or the catheter 36 are identified, the processor implementing an abnormal operation procedure may control one or more of the flow control devices 71, 72, 73, 74, and 75 to prevent undesired damage to any of the other unaffected elements caused by the infusate and/or the modification in operation of the accumulator 30 and/or the catheter 36.

The foregoing method descriptions and the process flow diagram are provided merely as illustrative examples and are not intended to require or imply that the blocks of the various aspects must be performed in the order presented. As will be appreciated by one of skill in the art the order of blocks in the foregoing aspects may be performed in any order. Words such as “thereafter,” “then,” “next,” etc. are not intended to limit the order of the blocks; these words are simply used to guide the reader through the description of the methods. Further, references to the diaphragm moving “up,” “down,” “upwardly,” and “downwardly” are merely for relating movements of the diaphragm in the orientation illustrated in the figures, and are not intended to limit the scope of the claims regarding a particular orientation of device or diaphragm with respect to the Earth. Further, any reference to claim elements in the singular, for example, using the articles “a,” “an” or “the” is not to be construed as limiting the element to the singular.

The various illustrative logical blocks, modules, circuits, and algorithm blocks described in connection with the aspects disclosed herein may be implemented as electronic hardware, computer software, or combinations of both. To clearly illustrate this interchangeability of hardware and software, various illustrative components, blocks, modules, circuits, and blocks have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

What is claimed is:

1. A method for determining whether a change in infusate volume within an implantable drug delivery device is normal, comprising:
obtaining, by a processor, a first sensor output from a bellows sensor of a parameter related to a volume of infusate within a bellows of the implantable drug delivery device;
obtaining, by the processor, a second sensor output from the bellows sensor of the parameter related to the volume of infusate within the bellows of the implantable drug delivery device;
determining, by the processor, a rate of change in the volume of the infusate within the bellows based on the first sensor output and the second sensor output;
determining, by the processor, whether the rate of change in a volume of infusate within the bellows satisfies a threshold criterion; and
initiating, by the processor, an abnormal infusate volume rate procedure in response to determining that the rate of change in the volume of infusate within the bellows does not satisfy the threshold criterion.

2. The method of claim 1, wherein:
determining whether the rate of change in the volume of infusate within the bellows does not satisfy the threshold criterion comprises determining whether the rate of change in the volume of infusate within the bellows exceeds the threshold criterion; and
initiating the abnormal infusate volume rate procedure comprises controlling, by the processor, a flow control device to shutoff flow of the infusate in response to determining that the rate of change in the volume of infusate within the bellows exceeds the threshold criterion.

3. The method of claim 1, wherein:
determining whether the rate of change in the volume of infusate within the bellows does not satisfy the threshold criterion comprises determining whether the rate of change in the volume of infusate within the bellows is less than the threshold criterion; and
initiating the abnormal infusate volume rate procedure comprises sending, by the processor via a wireless communication interface, a notification to an external device indicating that the implantable drug delivery device is operating in an abnormal state in response to determining that the rate of change in the volume of infusate within the bellows is less than the threshold criterion.

4. The method of claim 1, wherein initiating the abnormal infusate volume rate procedure comprises at least one of:
modifying, by the processor, one or more parameters of a current treatment regimen implemented on the implantable drug delivery device;
sending, by the processor via a wireless communication interface, a notification to an external device indicating that the implantable drug delivery device is operating in an abnormal state; or
determining, by the processor, whether a function of an element of the implantable drug delivery device is modified.

5. The method of claim 1, wherein the processor is a component of the implantable drug delivery device.

6. The method of claim 1, wherein the processor is included in a computing device external from the implantable drug delivery device.

7. The method of claim 1, further comprising:
determining, by the processor, an infusate flow rate based on an output received from a diaphragm sensor associated with an accumulator of the implantable drug delivery device; and
determining, by the processor, that the implantable drug delivery device is operating in an abnormal state based on the determined infusate flow rate and the volume of the infusate within the bellows of the implantable drug delivery device.

8. The method of claim 1, further comprising:
receiving, by the processor, an indication that the bellows is filled with infusate;
determining, by the processor, an initial parameter associated with the infusate in response to receiving the indication that the bellows is filled with infusate, wherein the initial parameter is determined based on a third output from the bellows sensor;
determining, by the processor, parameters associated with a treatment regimen based on the determined initial parameter; and
initiating, by the processor, the determined treatment regimen.

9. The method of claim 1, wherein the bellows sensor includes at least one of an electronically-based sensor, a strain gauge, a sensor configured to detect one or more of a position of the bellows, an orientation of the bellows, an expansion or compression state of the bellows, and a pressure associated with the bellows, a light based sensor including a light emitter and a light detector, a sensor configured to detect vapor and liquid of a two-phase liquid associated with the bellows, a flow meter configured to detect a flow of the infusate as it travels between the bellows and an accumulator, a sonically-based sensor, a capacitive displacement sensor, or an inductive sensor.

10. An implantable drug delivery device, comprising:
a bellows configured as a reservoir for infusate;
an accumulator coupled to the bellows and comprising a diaphragm chamber and a diaphragm that deflects within the diaphragm chamber to dispense the infusate to a patient;
a bellows sensor configured to measure a parameter related to a change in volume of the bellows;
a processor coupled to the bellows sensor and configured with processor-executable instructions to perform operations comprising:
obtaining a first sensor output from a bellows sensor of a parameter related to a volume of infusate within a bellows of the implantable drug delivery device;
obtaining a second sensor output from the bellows sensor of the parameter related to the volume of infusate within the bellows of the implantable drug delivery device;
determining a rate of change in the volume of the infusate within the bellows based on the first sensor output and the second sensor output;
determining whether the rate of change in a volume of infusate within the bellows does not meet a threshold criterion; and
initiating an abnormal infusate volume rate procedure in response to determining that the rate of change in the volume of infusate within the bellows does not meet the threshold criterion.

11. The implantable drug delivery device of claim 10, wherein the bellows sensor comprises at least one of an electronically-based sensor, a strain gauge, a sensor configured to detect one or more of a position of the bellows, an orientation of the bellows, an expansion or compression state of the bellows, and a pressure associated with the bellows, a light based sensor including a light emitter and a light detector, a sensor configured to detect vapor and liquid of a two-phase liquid associated with the bellows, a flow meter configured to detect a flow of the infusate as it travels between the bellows and an accumulator, a sonically-based sensor, a capacitive displacement sensor, or an inductive sensor.

12. The implantable drug delivery device of claim 10, wherein the bellows sensor comprises at least one of a strain gauge on a surface of the bellows or a capacitive displacement sensor.

13. The implantable drug delivery device of claim 10, wherein the bellows sensor comprises a light-based sensor configured to detect a change in a light signal related to a change in volume of the bellows.

14. The implantable drug delivery device of claim 10, wherein the bellows sensor comprises a sonically-based sensor configured to detect a change in sonic signals related to the volume of the bellows.

15. The implantable drug delivery device of claim 10, further comprising a flow control device within an infusate flow path within the implantable drug delivery device, wherein the processor is configured with processor-executable instructions to perform operations such that initiating the abnormal infusate volume rate procedure comprises controlling the flow control device to shutoff flow of the infusate.

16. The implantable drug delivery device of claim 10, wherein the processor is configured with processor-executable instructions to perform operations such that initiating the abnormal infusate volume rate procedure comprises at least one of:
modifying one or more parameters of a current treatment regimen implemented on the implantable drug delivery device; or
determining whether a function of an element of the implantable drug delivery device is modified.

17. The implantable drug delivery device of claim 10, further comprising a wireless communication transceiver coupled to the processor, wherein the processor is configured with processor-executable instructions to perform operations such that initiating the abnormal infusate volume rate procedure comprises:
sending an abnormal state notification to an external device using the wireless communication transceiver.

18. The implantable drug delivery device of claim 10, wherein the processor is configured with processor-executable instructions to perform operations further comprising:
receiving an indication that the bellows is filled with infusate;
determining an initial parameter associated with the infusate in response to receiving the indication that the bellows is filled with infusate, wherein the initial parameter is determined based on a third sensor output from the bellows sensor;
determining parameters associated with a treatment regimen based on the determined initial parameter; and
initiating, by the processor, the determined treatment regimen.

19. An implantable drug delivery device, comprising:
a bellows configured as a reservoir for infusate;
means for obtaining a first measure and a second measure of volume of infusate in the bellows;
means for determining a rate of change in the volume of the infusate within the bellows based on the first sensor output and the second sensor output;
means for determining whether the rate of change in a volume of infusate within the bellows does not meet a threshold criterion; and
means for performing an abnormal infusate volume rate procedure in response to determining that the rate of change in the volume of infusate within the bellows does not meet the threshold criterion.

20. The implantable drug delivery device of claim 19, wherein means for performing an abnormal infusate volume rate procedure comprises means for shutting off flow of infusate to a patient.

21. The implantable drug delivery device of claim 19, wherein means for obtaining a first measure and a second measure of volume of infusate in the bellows comprises at least one of an electronically-based sensor, a strain gauge, a sensor configured to detect one or more of a position of the bellows, an orientation of the bellows, an expansion or compression state of the bellows, and a pressure associated with the bellows, a light based sensor including a light emitter and a light detector, a sensor configured to detect vapor and liquid of a two-phase liquid associated with the bellows, a flow meter configured to detect a flow of the infusate as it travels between the bellows and an accumulator, a sonically-based sensor, a capacitive displacement sensor, or an inductive sensor.

22. A method for determining a volume of infusate within an implantable drug delivery device, comprising:
obtaining, by a processor, a sensor output from a bellows sensor of a parameter related to a volume of infusate within a bellows of the implantable drug delivery device;
determining, by the processor, whether the volume of infusate within the bellows satisfies a threshold criterion based on the parameter related to the volume of infusate within the bellows; and
initiating, by the processor, an abnormal infusate volume procedure in response to determining that the volume of infusate within the bellows does not satisfy the threshold criterion.
